(19) United States
(12) Patent Application Publication
(10) Pub. No.: US 2008/0207545 A1
(43) Pub. Date: Aug. 28, 2008

(54) Methods and Compositions for Treating 5Alpha-Reductase Type 1 and Type 2 Dependent Conditions

(76) Inventors: Glenn D. Hoke, Mt. Airy, MD (US); Thomas P. Nigra, Washington, DC (US)

Correspondence Address: MORGAN LEWIS & BOCKIUS LLP, 1111 PENNSYLVANIA AVENUE NW, WASHINGTON, DC, 20004, US

(21) Appl. No.: 11/885,384

(22) PCT Filed: Oct. 20, 2004
(86) PCT No.: PCT/US04/34510

§ 371 Date: Apr. 18, 2008

Related U.S. Application Data
(60) Provisional application No. 60/512,689, filed on Oct. 21, 2003.
(60) Provisional application No. 60/545,146, filed on Feb. 18, 2004.

(51) Int. Cl. A61K 31/7088; A61P 17/00; A61P 35/04; A61P 13/00

(52) U.S. Cl. 514/44

(57) ABSTRACT

The invention relates generally to the use of anti-sense oligonucleotides, small interfering RNA, and ribozymes to modulate expression of the human steroid 5α-reductase gene and thereby modulate levels of dihydrotestosterone (DHT). Elevated levels of DHT are associated with various disorders including, but not limited to, skin diseases, hair loss, hirsuitism, and benign prostatic hyperplasia. The invention specifically relates to formulations of these anti-sense oligonucleotides, small interfering RNA, and ribozymes for administration to treat and prevent disorders

### 1. RELATED APPLICATIONS

This application claims the benefit U.S. Provisional Application No. 60/512,689, filed Oct. 21, 2003 and U.S. Provisional Application No. 60/545,146, filed Feb. 18, 2004, the entirety of each of which is incorporated herein by reference.

### 2. FIELD OF THE INVENTION

The invention relates generally to the use of anti-sense oligonucleotides, small interfering RNA, and ribozymes to modulate expression of the human steroid 5α-reductase gene and thereby modulate levels of dihydrotestosterone (DHT). Elevated levels of DHT are associated with various disorders including, but not limited to, skin diseases, hair loss, hirsuitism, and benign prostatic hyperplasia. The invention specifically relates to formulations of these anti-sense oligonucleotides, small interfering RNA, and ribozymes for administration to treat and prevent disorders

### 3. BACKGROUND OF THE INVENTION

### 3.1 Human Steroid 5α-Reductase

Two isozymes of 5α-reductase, type 1 and type 2, have been described in man (the designation type 1 and type 2 reflects the chronological order in which the genes were isolated). (See e.g., Andersson and Russell, Structural and biochemical properties of cloned and expressed human and rat steroid 5α-reductases. Proc Natl Acad. Sci. USA, 87: 3640-4, 1990; see also Andersson et al., Deletion of Steroid 5 alpha-Reductase 2 Gene in Male Pseudohermaphroditism. Nature, 354: 159-61, 1991). In addition to structural differences, the two isozymes differ with respect to their biochemical properties, expression patterns, genetics, and pharmacology. (See e.g. Andersson et al., 1991, supra; see also Jenkins et al., Genetic and Paramacologic Evidence For More Than One Human Steroid 5α-Reductase. J. Clin. Inv., 89: 293-300, 1992). For example, 5α-reductase type 1 is found in sebaceous glands (e.g., in the prostate and skin), and 5α-reductase type 2 is found, for example, in the prostate and hair follicles. Elucidation of the tissue distribution and the roles that the two steroid 5α-reductase isozymes play in androgen action is currently the subject of intense research.

The locus name of steroid 5α-reductase type 1 is SRD5α1. This gene maps to the short arm of chromosome 5 in the p15 band. The locus name for steroid 5α-reductase type 2 is SRD5α2 and maps to the 2p23 band of chromosome 2. In addition to these genes there also appears to be an inactive, processed pseudogene for steroid 5α-reductase type 1, designated SRD5αP1, which maps to the q24-qter region of the X chromosome (Jenkins et al., Characterization and Chromasomal Mapping of Human Steroid 5α-Reductase Gene and Pseudogene and Mapping of the Mouse Homologue. Genomics, 11: 1102-1112, 1991; Thigpen and Russell, Four Amino Acid Segment in Steroid 5a-Reductase 1 Confers Sensitivity to Finasteride, A Competitive Inhibitor. J. Biol. Chem., 267: 8577-8583, 1992).

Genomic clones containing overlapping portions of steroid 5α-reductase type 1 and steroid 5α-reductase type 2 have been isolated and sequenced. Based on sequence analysis both genes appear to be at least 20 Kb in length and have 5 exons interrupted by 4 introns in exactly the same positions. The similarities in gene structure have led to the suggestion that the two genes arose from a duplication of an ancestral gene followed by divergence leading to separate physiological roles (Jenkins et al., 1991 supra; Thigpen and Russell, 1992, supra).

The two steroid 5α-reductase genes appear to have similar promoters and respond to the same transcription factors, however it is not clear whether the two isoforms are co-regulated under all physiological conditions. Current evidence suggests that the expression of the two isozymes varies in different tissues and cell types. Recent pharmacological evidence suggests that the expression of steroid 5α-reductase might be regulated such that the inhibition of one isozyme may lead to the up-regulation of the other isozyme (Hirsch et al., A Selective, Non-steroidal Inhibitor of Human Steroid 5α-Reductase Type 1. Proc. Natl. Acad. Sci., USA, 90(11): 5277-81, 1993). The enzyme steroid 5α-reductase type 1 shows approximately 50% homology at the primary amino acid sequence level to steroid 5α-reductase type 2; however, their hydropathy plots are nearly identical implying that higher order structures are highly conserved (Andersson et al., 1991, supra). The structural similarities between the two isozymes probably reflects constraints that are necessary for catalytic activity.

Enzymatic activity requires the presence of NADPH as a cofactor for reduction of testosterone to dihydrotestosterone. Recent reports suggest that the association of NADPH with the enzyme acts allosterically to induce a conformational change in the three dimensional structure of the protein thereby allowing the substrate to gain access to the catalytic site (Metcalf et al, Potent Inhibition Of Human Steroid 5α-Reductase (EC 1.3.1.30) by 3-Androstene-3-Carboxylic Acids. Bioogranic Chem., 1: 372-376, 1989). The pH optimum for steroid 5α-reductase type 1 is relatively broad (pH 6-8) which is in sharp contrast to steroid 5α-reductase type 2 that exhibits maximum activity, in vitro, at pH 5.5. (Jenkins et al., 1992, supra). Pharmacological studies have indicated that the two isozymes exhibit different sensitivities to inhibitors (Jenkins et al., 1992, supra).

3.1.1 5α-Reductase Type 1

The steroid 5α-reductase type 1 gene (SEQ ID NO: 1) encodes a protein with a molecular weight of about 29 kDa. The enzyme is highly hydrophobic and contains only 13% charged amino acid residues (Andersson and Russell, 1990, supra; Andersson et al., 1991, supra; Andersson et al, Expression Cloning And Regulation Of Steroid 5 Alpha-Reductase, An Enzyme Essential For Male Sexual Differentiation. J Biol Chem, 264: 16249-55, 1989). Transcription of steroid 5α-reductase type 1 produces a messenger RNA of approximately 2.1 kb. Originally, the gene was cloned from cDNA copies by expression cloning and PCR. The resulting clone has been fully sequenced and shows complete identity to the sequence of the exons derived from the genomic clone (Andersson et al., 1991, supra; Jenkins et al., 1991, supra; Thigpen and Russell, 1992, supra; Andersson et al., 1989, supra). The message for the steroid 5α-reductase type 1 gene is capped at the 5′ end and polyadenylated at the 3′ end. The inferred polyadenylation sites have been identified based on the sequence of the cDNA clone that contained part of the poly A tail.

3.1.2 5α-Reductase Type 2

The function of steroid 5α-reductase type 2 (SEQ ID NO: 28) is defined by its deficiency in male pseudohermaphrodism, the exact role of steroid 5α-reductase type 1 in androgen physiology is not clearly understood (See id; see also Jenkins et al., 1992, supra). Recent molecular genetic evidence has suggested that steroid 5α-reductase type 2 is responsible for the differentiation of the embryonic external genitalia and prostate (Andersson et al., 1991, supra). Biochemical studies have also shown that steroid 5α-reductase type 2 represents the major form of the enzyme in the adult prostate (Andersson and Russell, 1990, supra; Jenkins et al., 1992, supra). However, evidence from recent studies has suggested that the prostate also has the capacity to express steroid 5α-reductase type 1 (Hirsch et al., 1993, supra). The 4-azasteroid, finasteride (MK-906, Proscar®) is a relatively poor inhibitor (Ki≧300 nM) of steroid 5α-reductase type 1 (Andersson and Russell, 1990, supra). In contrast, finasteride inhibits steroid 5α-reductase type 2 at low nanomolar concentrations (Ki=3-5 nM). Recently, a new series of inhibitors has been identified that preferentially inhibits steroid 5α-reductase 1 at a Ki of ˜11 nM (Neubauer et al., LY191704 inhibits type I steroid 5 alpha-reductase in human scalp. J Clin Endocrinol Metab., (1996) 81(6):2055-60).

### 3.2 Acne Vulgaris

Androgenic stimulation of the sebaceous glands is required for the development of acne (Sheehan-Dare et al., 1988, supra). The severity of acne is related to the rate of sebum excretion which is reported to be under hormonal control. Sebaceous follicles involved in acne are characterized by the accumulation of abnormally desquamated corneocytes and excess sebum, which together form the microcomedo. This environment provides ideal growth conditions for Propionibacteriun acnes. The levels of P. acnes found in microcomedos range from low to very high. A reduction in the level of sebum should alter the microenvironment and, hence, reduce the ability of the P. acnes to flourish. The androgenic basis for the disease is supported by the observation that anti-androgenic therapy has been effectively employed for the treatment of acne. Since the occurrence of acne is not related to an increase in circulating androgen levels, it has been postulated that the local production of DHT may be an etiological factor in the development of acne. Specifically, the severity of the disease appears to be related to the capacity of the skin to metabolize T to DHT. This observation is consistent with the finding that steroid 5α-reductase activity in skin is reported to be increased in acne subjects (Hay and Hodgins, Metabolism Of Androgens By Human Skin In Acne. Br. J. Dermatol., 91: 123-133, 1974; Lookingbill et al., Tissue Production Of Androgens In Women With Acne. J. Am. Acad. Dernatol., 12: 1985; Sansone and Reisner, Differential Rates Of Conversion Of Testosterone To Dihydrotestosterone In Acne And In Normal Human Skin-A Possible Pathogenic Factor In Acne. J. Invest. Dermatol., 56: 366-72, 1971). As with other androgen-dependent disorders, the inhibition of steroid 5α-reductase biosynthesis should be a safe and effective means for treating acne.

### 3.3 Benign Prostatic Hyperplasia (BPH)

In humans, the prostate gland undergoes hyperplastic and neoplastic changes that increase in frequency with advancing age (Durocher et al, Tyrosine Protein Kinase Of Human Hyperplastic Prostate And Carcinoma Cell Lines PC3 And DU145. Cancer Res., 49: 4818-4823, 1989; Walsh, Benign Prostatic Hyperplasia: Etiological Considerations (eds.), Prostatic Diseases, pp. 1-6, New York, N.Y.: Alan R. Liss Inc, 1976). Following a growth phase, which occurs at puberty, the weight of the prostate remains relatively constant (at approximately 20 grams) until the fifth decade when, in about 75-80 percent of men, the gland undergoes a second round of growth (Clark et al., Mechanisms Of Steroid Hormone Action In: J. D. Wilson and D. W. Foster (eds.), Textbook of Endocrinology, pp. 67-68, Philadelphia, Pa.: W.B. Saunders Company, 1985). The resulting non-malignant prostatic hyperplasia (BPH) leads to an enlargement of the gland that can result in the development of obstructive and irritative urinary voiding symptoms along with a predisposition to urinary tract infections. (Durocher et al., 1989, supra; Smith et al., Benign Prostatic Hyperplasia. Postgrad. Med., 83: 79-85, 1988.).

For over 100 years BPH has been treated surgically resulting in prostatectomy being the most common surgery performed in men over 65 years of age (Stimson and Finn, Benign Prostatic Hyperplasia And Its Treatment. J. Gen. Int. Med., 5: 153-165, 1990). In 1985 alone there were 367,000 prostatectomies performed in the United States at an approximate cost of three billion dollars. Furthermore, recent evidence suggests that over 20 percent of men living to 80 years of age will undergo surgical resection of the prostate. Although prostatic surgery has been greatly refined, it still remains a major cause of morbidity in elderly men (Matzkin and Braf, Endocrine Treatment Of Benign Prostatic Hypertrophy Current Concepts. Urology, 37: 1-16, 1991). Transurethral resection of the prostate (TURP) is normally conducted under spinal anesthesia and has a mortality rate of approximately one percent. Recurrence of urinary obstruction can occur in up to 20% of patients who have undergone surgery.

Histologic examination of prostatic tissue from patients with BPH reveals regions of focal atrophy, that are characterized by shrinking glands, chronic inflammation, and stromal fibrosis. Although the disease exists in several forms, the most common is characterized by a five-fold increase in the prostatic stroma and a two-fold increase in glandular tissue (MacIndoe et al., Comparative Studies Of 5 Alpha-Reductase Inhibitors Within MCF-7 Human Breast Cancer Cells. J Steroid Biochem, 20:1095-100, 1984; McNeal, The Anatomic Heterogeneity Of The Prostate, Models for Prostate Cancer, pp. 149-160, New York, N.Y.: Alan R. Liss, 1980; McNeal, Normal Histology Of The Prostate. Am. J. Surg. Pathol., 12: 619, 1988). Within the stroma there is also an accumulation of connective tissue and an activation of smooth muscle elements (Bruengger et al., Smooth Muscle Cell Of The Canine Prostate In Spontaneous Benign Hyperplasia, Steroid Induced Hyperplasia And Estrogen Or Tamoxifen Treated Dogs. J. Urol., 130: 1208-1210, 1983).

Although the etiology of BPH is unclear, there is strong evidence supporting a role for androgens in the development of prostatic hyperplasia (Walsh, 1976, supra). Male sexual tissues show a profound dependence on the continuous presence of androgens for normal growth and function (Walsh, 1976, supra; Djoseland et al., 5α-Reductase Activity In Stromal And Epithelium Of Rat Prostate And Epididymis. A Contribution To Elucidation Of The Mechanism For The Development Of Hyperplastic Growth Of Prostatic Tissue. Acta Endo., 103: 273-281, 1983; Hierowski et al., The Partial Dependency Of Hyman Prostatic Growth Factors On Steroid Hormones In Stimulating Thymidine Incorporation Into DNA. J. Urol., 138: 909-912, 1987; Kyprianou and Isaacs, Identification Of A Cellular Receptor For Transforming Growth Factor In Rat Ventral Prostate And Its Negative Regulation By Androgens. Endocrinology, 123: 2124-2131, 1988). Testosterone (T), is produced by the testes and is widely distributed whereas dihydrotestosterone (DHT) is produced locally from testosterone through a NADPH-dependent reaction that is catalyzed by the membrane-bound enzyme steroid 5α-reductase (EC 1.3.995) (Wilson, Handbook of Physiology: Endocrinoloy, Vol. 5, pp. 491-508, Washington: Am. Physiol. Soc., 1975). Testosterone and DHT have both overlapping and distinct roles in androgen physiology. In the embryo, T acts to promote the masculinization of the wolffian ducts leading to formation of the vas deferens, epididymis, and seminal vesicles. DHT acts to induce formation of the external genitalia and the prostate (Griffin and Wilson eds., The Metabolic Basis of Inherited Diseases, pp. 1919-1944, New York: McGraw-Hill, 1989).

In most mammalian species the production of DHT occurs locally within the cells of androgen-responsive tissues. Early studies revealed that, in hyperplastic glands, the concentration of the androgen dihydrotestosterone (DHT) is greater than that observed in normal prostatic tissue (Siiteri et al, Dihydrotestosterone In Prostatic Hypertrophy. J. Clin. Inv., 49:1737-1745, 1970.). Furthermore, the highest concentration of DHT appears to occur in the periurethral region. It is within this region that cellular hyperplasia is most often observed. The elevation of prostatic DHT concentrations has been attributed to an increase in prostatic steroid 5α-reductase activity (Bruchovsky and Lieskovsky, Increased Ratio Of 5alpha-Reductase:3alpha(Beta)-Hydroxysteroid Dehydrogenase Activities In The Hyperplastic Human Prostate. J. Endo., 80: 289-301, 1979). An examination of steroid 5α-reductase activity expressed by normal and BPH-derived prostatic tissue revealed that, while the Kₘ in hyperplastic tissue was unchanged, the Vₘₐₓ of the enzyme increased approximately 10-fold suggesting that the disease is associated with an increase in the expression of the protein (Hudson et al, Studies On The Nuclear 5α-Reductase Of Hyman Prostatic Tissue: Comparison Of Enzyme Activities In Hyperplastic, Malignant, And Normal Tissue. Can. J. Cell Bio., 61: 750-755, 1982). The increase in stromal steroid 5α-reductase activity appears to be an age-related phenomenon (Hochstrate et al., Effects Of Aging On Kinetic Parameters Of 5α-Reductase In Epithelium And Stroma Of Normal And Hyperplastic Human Prostate. J. Clin. Endocrinol. Metab., 67: 979-985, 1988).

Based on the importance of androgens, particularly DHT, in prostate physiology recent attempts at treating BPH have sought to block androgen action through the inhibition of steroid 5α-reductase activity (U.S. Pat. No. 4,760,071). This therapeutic approach represents an attempt at treating the underlying mechanisms that lead to the development of prostatic hyperplasia. To date, a number of steroid 5α-reductase inhibitors have been described, (Metcalf et al., Inhibitors Of Steroid 5 Alpha-Reductase In Benign Prostatic Hyperplasia, Male Pattern Baldness And Acne. Trends Pharmacol Sci, 10: 491-5, 1989; Metcalf et al., 1989, supra; Petrow et al., Prostatic Cancer. I. 6-Methylene-4-Pregnen-3-Ones As Irreversible Inhibitors Of Rat Prostatic Delta 4-3 Ketosteroid 5 Alpha-Reductase. Steroids, 38: 121-40, 1981; Petrow et al., Ihibition Of Prostatic Growth In Rats By 6-Methylene-4-3,20-Dione. J Endocrinol, 95: 311-3, 1982; Marts et al., A Comparison Of The Effects Of Castration And 6-Methylene Progesterone, A 5 Alpha-Reductase Inhibitor, On The Rat Ventral Prostate. Biochem Cell Biol, 65: 626-34, 1987; Stoner, The Clinical Development Of A 5α-Reductase Inhibitor, Finasteride. J Steroid Biochem Mol Biol, 37: 375-8, 1990). Of the existing compounds, finasteride (MK-906, Propecia®, Proscar®) has undergone the most extensive clinical trials in patients with BPH. The results of these trials indicate that the compound causes a 65 percent decrease in plasma DHT levels while circulating levels of T are maintained or slightly increased (Stoner, 1990, supra) As a result, libido and muscular strength and other presumably testosterone-dependent characteristics are not affected by steroid 5α-reductase inhibition. The inhibition of steroid 5α-reductase by finasteride is accompanied by a decrease in prostatic volume and an increase in maximal urinary flow rates in approximately 30 percent of patients with BPH (Stoner, 1990, supra).

Although the predominate form of steroid 5α-reductase in the prostate appears to be type 2, recent pharmacologic evidence has demonstrated that cultured prostate cells are capable of expressing steroid 5α-reductase type 1 (Hirsch et al., 1993).

### 3.4 Androgenic Alopecia

The majority of facial and body hair growth is stimulated by androgens. However, the growth of scalp hair has been shown to be inhibited by DHT in individuals who exhibit a hereditary predisposition to baldness (Ebling, Steroids And The Skin: A General Review. Biochem Soc Trans, 4: 597-602, 1976; Lucky, The Paradox Of Androgens And Balding: Where Are We Now?, J Invest Dermatol, 91: 99-100, 1988; Brodland and Muller, Androgenetic Alopecia (Common Baldness). Cutis, 47: 173-6, 1991). The phenotypic expression of baldness does not occur in the absence of androgens. Androgenic alopecia or common baldness represents 99 percent of all cases of hair loss (Brodland and Muller, 1991, supra). The incidence in men in their third to fifth decade is approximately 47 percent and increases with increasing age. In premenopausal women the incidence is relatively low (9 percent). However, by the sixth decade the occurrence of baldness in women has been estimated at 39 percent.

The mechanism through which androgens, in particular DHT, function to regulate the biology of hair is by modulation of the hair growth cycle (Ebling, 1976, supra; Bergfeld and Redmond, Androgenic Alopecia. Dermatol Clin, 5: 491-500, 1987). The effects of DHT on hair growth appear to be related to local rather than systemic levels of the hormone in that the capacity of scalp skin from balding individuals to convert T to DHT is greater than that observed in the scalp of non-balding individuals (Ebling, 1976, supra; Lucky, 1988, supra; Schweikert and Wilson, 1974, Regulation Of Human Hair Growth By Steroid Hormones. I. Testerone Metabolism In Isolated Hairs. J Clin Endocrinol Metab, 38: 811-9, 1974). Additional evidence supporting the notion that balding is related to an over production of DHT in the scalp is provided by studies with the aza-steroid 4-MA (17β-N,N,-diethylcarbamoyl-4-methyl-4-aza-5α-androstan-3-one) in the stumptail macaque. These monkeys undergo an age-related hair loss similar to that observed in man (Rittmaster et al., The Effects Of N,N-Diethyl-4-Methyl-3-Oxo4-Aza-5 Alpha-Androstane-17 Beta-Carboxamide, A 5 Alpha-Reductase Inhibitor And Antiandrogen, On The Development Of Baldness In The Stumptail Macaque. J Clin Endocrinol Metab, 65: 188-93, 1987). In this model system, monkeys treated with the compound grew significantly more hair than did the untreated monkeys or those treated with vehicle alone (Rittmaster et al., 1987, supra). Preliminary biochemical evidence suggests that, in humans, the scalp expresses predominately steroid 5α-reductase 1 (Itarni, Journal of Investigative Dermatology, 95: 57-60, 1991). Recent reports have suggested that 4-MA is capable of inhibiting both steroid 5α-reductase isozymes (Andersson and Russell, 1990, supra).

### 3.5 Hirsutism

Hirsutism effects eleven percent of women and is characterized by an excessive growth of coarse, terminal body hair in a male hair growth pattern (Bergfeld and Redmond, Hirsutism. Dermatolo. Clin., 5: 501-507, 1987; Ehrmann and Rosenfield, Clinical Review 10: An Endocrinologic Approach To The Patient With Hirsutism. J Clin Endocrinol Metab, 71: 1-4, 1990). Specifically, there is a conversion of fine vellus hair to terminal coarse hair on the chest, face, shoulders, back, and abdomen. As is the case in men, this pattern of hair growth is reported to be androgen-dependent. Women with hirsutism frequently (50-85%) exhibit some form of hyperandrogenemia along with a premature shedding and thinning of scalp hair.

As is the case in other androgen-dependent conditions, the effects of androgens on hair growth patterns in hirsutism appear to be related to an increase in the local production of DHT. Furthermore, there is evidence suggesting that in women expressing the condition, the conversion of T to DHT is increased in androgen target tissues (Lobo et al., Production Of 3alpha-Androstenediol Glucuronide In Human Genital Skin. J. Clin. Endocrinol. Metab, 65: 711-714, 1987). In skin, steroid 5α-reductase activity has been localized in the sebaceous and sweat glands which are androgen responsive (Ehrmann and Rosenfield, 1990, supra). Recent reports have suggested that, in androgen-dependent tissues, the expression of steroid 5α-reductase activity is regulated by androgens (George et al., Feed-Forward Control Of Prostate Growth: Dihydrotestosterone Induces Expression Of Its Own Biosynthetic Enzyme, Steroid 5 Alpha-Reductase. Proc Natl Acad Sci USA, 88: 8044-7, 1991).

### 4. SUMMARY OF THE INVENTION

The invention relates to compositions and methods for treating and preventing disorders related to 5α-reductase type 1 and type 2.

In one embodiment, the invention encompasses compositions comprising a therapeutically or prophylactically effective amount of anti-sense oligonucleotides, small interfering RNA (“siRNA”), or ribozymes capable of inhibiting or mitigating the conversion of testerone to dihydrotestosterone.

In another embodiment, the invention encompasses compositions comprising a therapeutically or prophylactically effective amount of one or more anti-sense oligonucleotides of the invention that reduce the expression of human steroid 5α-reductase type 1 mRNA or are capable of hybridizing to the mRNAs for human steroid 5α-reductase type 1 in animals. In yet another embodiment, the invention encompasses compositions comprising a therapeutically or prophylactically effective amount of one or more anti-sense oligonucleotides of the invention that reduce the expression of human steroid 5α-reductase type 2 mRNA or are capable of hybridizing to the mRNAs for human steroid 5α-reductase type 2 in animals. In another embodiment, the invention encompasses compositions comprising a therapeutically or prophylactically effective amount of one or more anti-sense oligonucleotides of the invention that reduce the enzymatic activity of human steroid 5α-reductase type 1 in an animal. In another embodiment, the invention encompasses compositions comprising a therapeutically or prophylactically effective amount of one or more anti-sense oligonucleotides of the invention that reduce the enzymatic activity of human steroid 5α-reductase type 2 in an animal.

In yet another embodiment, the invention encompasses compositions comprising a therapeutically or prophylactically effective amount of small interfering RNA of the invention that reduce the expression of human steroid 5α-reductase type 1 mRNA or are capable of hybridizing to the mRNAs for human steroid 5α-reductase type 1 in animals. In another embodiment, the invention encompasses compositions comprising a therapeutically or prophylactically effective amount of siRNA of the invention that reduce the expression of human steroid 5α-reductase type 2 mRNA or are capable of hybridizing to the mRNAs for human steroid 5α-reductase type 2 in animals. In yet another embodiment, the invention encompasses compositions comprising a therapeutically or prophylactically effective amount of siRNA of the invention that reduce the enzymatic activity of human steroid 5α-reductase type 1 in an animal. In another embodiment, the invention encompasses compositions comprising a therapeutically or prophylactically effective amount of siRNA of the invention that reduce the enzymatic activity of human steroid 5α-reductase type 2 in an animal.

In still another embodiment, the invention encompasses compositions comprising a therapeutically or prophylactically effective amount of a ribozyme of the invention that reduce the expression of human steroid 5α-reductase type 1 mRNA or are capable of hybridizing to the mRNAs for human steroid 5α-reductase type 1 in animals. In another embodiment, the invention encompasses compositions comprising a therapeutically or prophylactically effective amount of a ribozyme of the invention that reduce the expression of human steroid 5α-reductase type 2 mRNA or are capable of hybridizing to the mRNAs for human steroid 5α-reductase type 2 in animals. In yet another embodiment, the invention encompasses compositions comprising a therapeutically or prophylactically effective amount of a ribozyme of the invention that reduces the enzymatic activity of human steroid 5α-reductase type 1 in an animal. In another embodiment, the invention encompasses compositions comprising a therapeutically or prophylactically effective amount of a ribozyme of the invention that reduces the enzymatic activity of human steroid 5α-reductase type 2 in an animal.

The compositions comprising a therapeutically or prophylactically effective amount of an oligonucleotide or a ribozyme of the invention are useful in treating or preventing disorders including, but not limited to, skin inflammation, disorders related to sebum secretion or excess sebum secretion, disorders related to sebum production or excess sebum production, steatoma, cystic acne, excess keratin production, comedones, papules, pustules, milia, seborrheic dermatitis, dandruff, seborrheic eczema, infantile seborrheic eczema, seborrheic keratosis, rosacea, perioral dermatitis, sebaceous cysts, acne vulgaris, oily skin, seborrheic wart, senile wart, basil cell papilloma, hirsutism, dermatosis paulosa nigra, benign prostatic hyperplasia, prostate cancer, urinary incontinence, androgenic alopecia, and male pattern baldness. Without being limited by theory it is believed that in one mode of action the compositions of the invention are useful in inhibiting the conversion of testosterone to dihydrotestosterone.

In another embodiment, the invention encompasses methods of inhibiting the conversion of testosterone to dihydrotestosterone in a patient in need thereof, which comprise administering to said patient a composition comprising a therapeutically or prophylactically effective amount of one or more anti-sense oligonucleotides, siRNA, or ribozymes of the invention.

In another embodiment, the invention encompasses methods of treating or preventing skin inflammation, disorders related to sebum secretion or excess sebum secretion, disorders related to sebum production or excess sebum production, steatoma, cystic acne, excess keratin production, comedones, papules, pustules, milia, seborrheic dermatitis, dandruff, seborrheic eczema, infantile seborrheic eczema, seborrheic keratosis, rosacea, perioral dermatitis, sebaceous cysts, acne vulgaris, oily skin, seborrheic wart, senile wart, basil cell papilloma, hirsutism, dermatosis paulosa nigra, benign prostatic hyperplasia, prostate cancer, urinary incontinence, androgenic alopecia, and male pattern baldness in a patient in need thereof, which comprise administering to said patient a composition comprising a therapeutically or prophylactically effective amount of one or more anti-sense oligonucleotides, siRNA, or ribozymes of the invention.

In another embodiment, the invention encompasses compositions comprising a therapeutically or prophylactically effective amount of one or more anti-sense oligonucleotides, siRNA, or ribozymes in combination with one or more additional therapeutic agents. The other therapeutic agent provides additive or synergistic value relative to the administration of an antisense oligonucleotide, siRNA, or ribozyme of the invention alone.

The invention further encompasses delivery vehicles that provide enhanced penetration of these compositions into the skin by the incorporation of a penetration enhancer including, but not limited to, ethyl alcohol, propylene glycol, glycerin, dimethyl isosorbide, polyethylene glycol ester, EDTA, panthethine, and a divalent cation, such as zinc.

### 5. BRIEF DESCRIPTION OF THE DRAWINGS

FIG. 1 shows the results of a trial in which groups of five subjects were treated twice daily with a topical formulation containing an anti-human steroid 5α-reductase type 1 (anti-HS5αR) anti-sense oligonucleotide (1 μM DPC1528), or a mis-sense or scrambled anti-sense oligonucleotide, or a control formulation for four weeks. The formulation containing the anti-sense oligonucleotide produced a 25% decrease in sebum production compared to the mis-sense and vehicle controls.

FIG. 2 shows the inhibition of sebum production following four weeks of twice daily treatment with formulations containing 1 μM (Study 1 and 2) or 5 μM (Study 3) anti-HS5αR anti-sense oligonucleotide (DPC1528). The formulation containing 1 uM anti-sense oligonucleotide produced a 24-27% decrease in sebum production and the formulation containing 5 μM produced a 33% decrease.

FIG. 3 shows the reduction in sebum production following intermittent treatment with anti-HS5αR anti-sense oligonucleotide (DPC1528). The formulation containing 1 μM anti-sense oligonucleotide was applied topically twice daily for two periods of four weeks each with no treatment for an intervening period of two weeks. The formulation containing the anti-sense oligonucleotide produced a 24% decrease in sebum production over the first four weeks, and a 20% decrease over the second four weeks.

FIG. 4 shows enhanced delivery of [³⁵S]-phosphorothioate oligonucleotide by a vehicle containing dimethyl isosorbide in human skin explants. Human skin explants treated one, two, or three times with a 7 uM concentration of oligonucleotide in a formula containing dimethyl isosorbide were tested for the ability of the compound to be transported into the sub-stratum corneum layers of skin. The oligonucleotide-containing compound was allowed to penetrate the skin for 24 hours and the capacity of the formula to enhance penetration was then measured.

FIG. 5 shows inhibition of steroid 5-α-reductase type II expression in COS cells expressing human 5-α-reductase-type II by DPC1676, a phosphorothioate modified 21-nucleotide antisense inhibitor and the failure of three scrambled, similar in composition but differing in linear sequence, or non-antisense phosphorothioate modified sequences to inhibit expression.

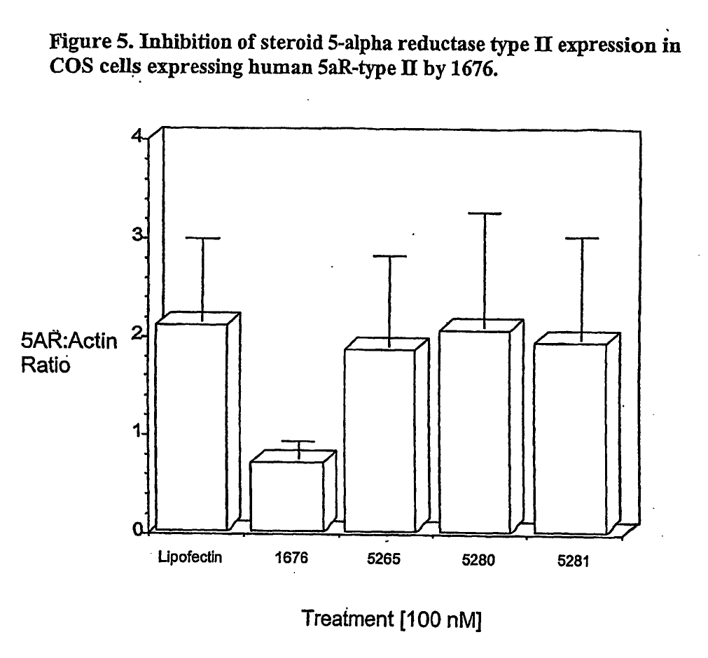

FIG. 6 shows the dose response inhibition of human steroid 5-α-reductase type II dose response by the 21-mer phosphoDPC 1676 and lack of a dose response by DPC1533, a scrambled sequence derived from DPC1676. The IC₅₀ for DPC1676 is ˜3 nM with maximum inhibition seen at ˜10 nM.

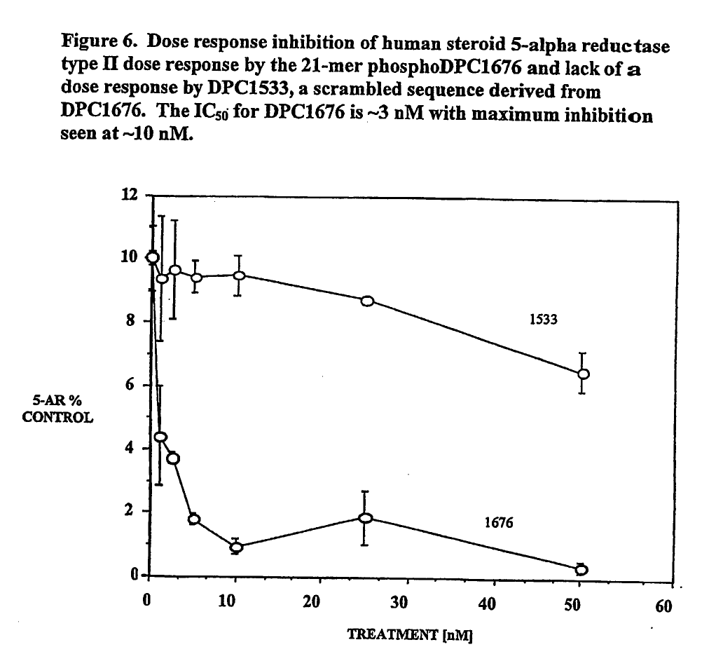

FIG. 7 shows antisense oligonucleotide DPC1676 targeting human steroid 5-α-reductase type II inhibits expression of the 5-α-reductase type II isoform by 80% compared to vehicle treated control cells when applied at 100 nM to COS cells expressing human 5aR-type II. Neither the scrambled control phosphorothioate oligomerPDC5265 nor the reverse complementary phosphorothioate oligonucleotide to DPC1676, DPC5277 did not reduce expression. However, co-administration of DPC1676 and DPC5277 resulted in binding of these two phosphorothioate oligomers to each other, reducing the ability of DPC1676 to bind to the mRNA for 5-α-reductase type II, and hence reduced the inhibition of expression of the protein typically observed when DPC1676 is administered alone. This data shows that DPC1676 is capable of inhibiting expression of human steroid 5-α-reductase type II by an antisense mechanism.

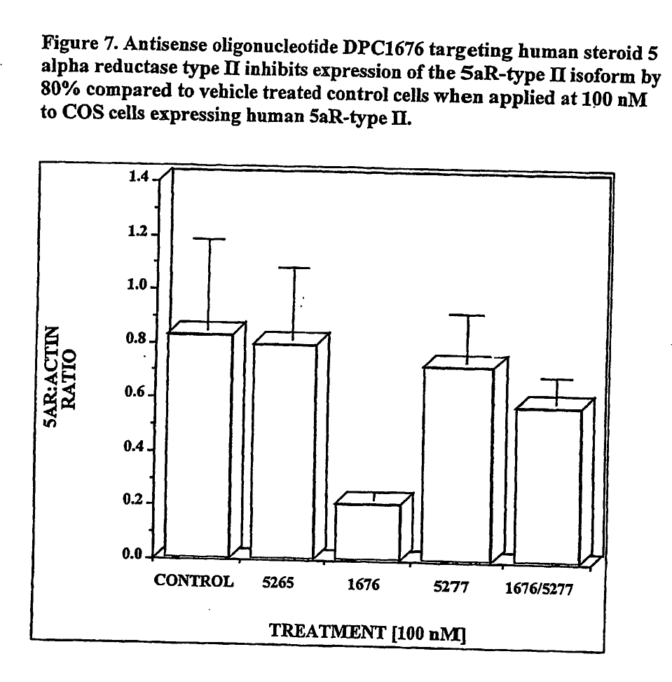

### 6. DETAILED DESCRIPTION

### 6.1 Definitions

As used herein, unless otherwise indicated, the term “animal(s)” refers to mammals, particularly humans.

As used herein, unless otherwise indicated, the terms “antisense” or “antisense oligonucleotide” refer to oligonucleotides or modified oligonucleotides that bind in a sequence specific manner to the pre-mRNA or mRNA of steroid 5α-reductase type 1 gene, or to the pre-mRNA or mRNA of steroid 5α-reductase type 2 gene. Antisense compositions may further include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation.

As used herein, unless otherwise indicated, the phrase “oligonucleotide of the invention” includes the terms “anti-sense oligonucleotide,” “oligonucleotide,” “oligodeoxynucleotide,” “oligodeoxyribonucleotide,” “nucleic acid-based compound,” “nucleic acid molecule,” “siRNA,” “ribozyme,” and “aptamer” and include oligomers and polymers of the biologically significant nucleotides, adenine, deoxyadenine, guanine, deoxyguanine, thymidine, uridine, cytosine and deoxycytosine, as well as oligomers and polymers that contain other novel nucleotides. These terms also include oligomers and polymers having one or more purine or pyrimidine moieties, sugar moieties, or internucleotide linkage(s) that have been chemically modified. These terms include any oligomers and polymers that are composed of nucleotides or nucleotides containing any modifications listed above which also contain bases or modified bases that are joined to sugar moieties in the alpha and not the beta configuration (known in the art as “alpha anomers”) or any oligonucleotide or polynucleotide that contains one or more of these modifications. The oligonucleotides can be linear or circular and include oligomers that are modified at the 5′-end, 3′-end, or anywhere in the middle of the chain. Modifications may also involve the backbone or may occur through the nucleobases with reporter groups. These reporter groups can be lipids, phospholipids, sugarlipids, etherlipids, peptides, ligands to known or unknown receptors or any other hydrophobic moiety that can enhance or regulate the cellular uptake or the targeting of the oligonucleotide to a particular cell type, including carrier groups that are attached to an oligonucleotide to enhance penetration across cellular membranes, which carrier groups include, but are not limited to, lipids, peptides, aliphatic groups (e.g., methylene, ethylene, propylene) or non-polar groups. The reporter groups can also be a cross-linking group that can form covalent linkages between the oligonucleotide and the targeted mRNA with or without biological or chemical activation. The sugar-phosphate backbone can be joined by 3′-5′ or 2′-5′ linkages. The backbone modifications of the oligonucleotides may include those known in the art including phosphotriesters, methylphosphonates, phosphodiesters or phosphorothioates and also such backbone modifications which are based on peptides or any other non-phosphate linkages that are currently being employed or might be used by those skilled in the art. These terms also include any oligomer or polymer that has nucleosides, whether natural or containing modifications, that are joined together in linkages that are not 3′-5′, such as 2′-3′ phosphodiester, 2′-5′ phosphodiester, or phosphorothioate linkages.

The term “oligonucleotide of the invention” also includes pharmaceutically acceptable salts, solvates, hydrates, clathrates, polymorphs and prodrugs thereof. In addition, the oligonucleotides of the invention may contain one or more chiral centers and/or double bonds and, therefore, exist as stereoisomers, such as double-bond isomers (i.e., geometric isomers), enantiomers, or diastereomers. According to the invention, the chemical structures depicted herein, and therefore the oligonucleotides of the invention, encompass all of the corresponding oligonucleotide's enantiomers and stereoisomers, that is, both the stereomerically pure form (e.g., geometrically pure, enantiomerically pure, or diastereomerically pure) and enantiomeric and stereoisomeric mixtures. Enantiomeric and stereoisomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically- or enantiomerically-pure intermediates, reagents, and catalysts by well known asymmetric synthetic methods. The terms “oligonucleotide of the invention” and “anti-sense oligonucleotide” are used interchangeably throughout and encompass each other.

As used herein and unless otherwise indicated, the term “compositions of the invention” refers to an oligonucleotide, siRNA, or ribozyme of the invention or pharmaceutically acceptable salts, solvates, hydrates, clathrates, polymorphs and prodrugs thereof and a pharmaceutically acceptable vehicle.

As used herein and unless otherwise indicated, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the invention is administered. Such pharmaceutical vehicles can be, for example, liquids, such as water and oils, including those of petroleum, animal, vegetable or synthetic origin, such as peanut oil, soybean oil, mineral oil, sesame oil and the like. The pharmaceutical vehicles can be saline, methyl cellulose, gum acacia, gelatin, starch paste, talc, keratin, colloidal silica, urea, and the like. In addition, auxiliary, stabilizing, thickening, lubricating and coloring agents may be used. When administered to a patient, the compositions of the invention and pharmaceutically acceptable vehicles are preferably sterile. Water is a preferred vehicle when the composition of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

As used herein and unless otherwise indicated, the phrase “pharmaceutically acceptable salt(s),” includes, but is not limited to, salts of acidic or basic groups that may be present in compounds used in the present compositions. Oligonucleotides included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, (i.e., salts containing pharmacologically acceptable anions), including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Oligonucleotides included in the present compositions that include an amino moiety may form pharmaceutically acceptable salts with various amino acids, in addition to the acids mentioned above. Oligonucleotides, included in the present compositions, that are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

As used herein and unless otherwise indicated, the term “pharmaceutically acceptable solvate” means an oligonucleotide of the invention that further includes a stoichiometric or non-stoichiometric amount of a solvent bound by non-covalent intermolecular forces. Preferred solvents are volatile, non-toxic, and/or acceptable for aistration to humans in trace amounts.

As used herein and unless otherwise indicated, the term “pharmaceutically acceptable hydrate” means an oligonucleotide of the invention that further includes a stoichiometric or non-stoichiometric amount of water bound by non-covalent intermolecular forces.

As used herein and unless otherwise indicated, the term “pharmaceutically acceptable clathrate” means an oligonucleotide of the invention that is in the form of a crystal lattice that contains spaces (e.g., channels) that have a guest molecule (e.g., a solvent or water) trapped within.

As used herein and unless otherwise indicated, the term “pharmaceutically acceptable polymorph” refers to an oligonucleotide of the invention that exists in several distinct forms (e.g., crystalline, amorphous), the invention encompasses all of these forms. Polymorphs are, by definition, crystals of the same molecule having different physical properties as a result of the order of the molecules in the crystal lattice. The differences in physical properties exhibited by polymorphs affect pharmaceutical parameters such as storage stability, compressibility and density (important in formulation and product manufacturing), and dissolution rates (an important factor in determining bio-availability). Differences in stability can result from changes in chemical reactivity (e.g., differential oxidation, such that a dosage form discolors more rapidly when comprised of one polymorph than when comprised of another polymorph) or mechanical changes (e.g., tablets crumble on storage as a kinetically favored polymorph converts to thermodynamically more stable polymorph) or both (e.g., tablets of one polymorph are more susceptible to breakdown at high humidity). As a result of solubility/dissolution differences, in the extreme case, some polymorphic transitions may result in lack of potency or, at the other extreme, toxicity. In addition, the physical properties of the crystal may be important in processing: for example, one polymorph might be more likely to form solvates or might be difficult to filter and wash free of impurities (i.e., particle shape and size distribution might be different between one polymorph relative to the other).

As used herein and unless otherwise indicated, the term “pharmaceutically acceptable prodrug” means a derivative of a compound that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise oligonucleotides, peptides, lipids, aliphatic and aromatic groups, or NO, NO₂, ONO, and ONO₂ moieties. Prodrugs can typically be prepared using well known methods, such as those described in Burger's Medicinal Chemistry and Drug Discovery, 172, 178, 949, 982 (Manfred E. Wolff ed., 5th ed. 1995), and Design of Prodrugs (H. Bundgaard ed., Elselvier, New York 1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxy-methyl, pivaloyloxymethyl, and pivaloyloxyethyl esters), lactonyl esters (such as phthalidyl and thiophthalidyl esters), lower alkoxyacyloxyalkyl esters (such as methoxycarbonyloxy-methyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, a amino acid amides, alkoxyacyl amides, and alkylaminoalkyl-carbonyl amides. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein and unless otherwise indicated, the term “therapeutically effective” refers to an amount of an oligonucleotide of the invention, siRNA of the invention, or ribozyme of the invention or a pharmaceutically acceptable salt, solvate, hydrate, clathrate, polymorph, or prodrug thereof able to cause an amelioration of a disease or disorder, or at least one discernible symptom thereof “Therapeutically effective” refers to an amount of an oligonucleotide of the invention, siRNA of the invention, or ribozyme of the invention or a pharmaceutically acceptable salt, solvate, hydrate, clathrate, polymorph, or prodrug thereof to result in an amelioration of at least one measurable physical parameter, not necessarily discernible by the patient. In yet another embodiment, the term “therapeutically effective” refers to an amount of an oligonucleotide of the invention, siRNA of the invention, or ribozyme of the invention or a pharmaceutically acceptable salt, solvate, hydrate, clathrate, polymorph, or prodrug thereof to inhibit the progression of a disease or disorder, either physically (e.g., stabilization of a discernible symptom), physiologically (e.g., stabilization of a physical parameter), or both. In yet another embodiment, the term “therapeutically effective” refers to an amount of an oligonucleotide of the invention, siRNA of the invention, or ribozyme of the invention or a pharmaceutically acceptable salt, solvate, hydrate, clathrate, polymorph, or prodrug thereof resulting in a delayed onset of a disease or disorder.

As used herein and unless otherwise indicated, the term “prophylactically effective” refers to an amount of an oligonucleotide of the invention, siRNA of the invention, or ribozyme of the invention or a pharmaceutically acceptable salt, solvate, hydrate, clathrate, polymorph, or prodrug thereof causing a reduction of the risk of acquiring a given disease or disorder. In one embodiment, the compositions of the invention are administered as a preventative measure to an animal, preferably a human, having a genetic predisposition to a disorder described herein. In another embodiment of the invention, the oligonucleotide of the invention, siRNA of the invention, or ribozyme of the invention or compositions comprising an oligonucleotide, siRNA of the invention, or ribozyme of the invention are administered as a preventative measure to a patient having a non-genetic predisposition to a disorder disclosed herein. Accordingly, the compositions of the invention may be used for the prevention of one disease or disorder and concurrently treating another (e.g., prevention of benign protatic hyperplasia, while treating urinary incontinence).

As used herein and unless otherwise indicated, the term “downstream” is used herein to indicate the 5′-3′ direction in a nucleotide sequence. Similarly, the term “upstream” indicates the 3′-5′ direction.

As used herein and unless otherwise indicated, the term “substantially complementary” is used herein to indicate that the oligonucleotide is capable of hybridizing to its mRNA target sequence.

As used herein and unless otherwise indicated, the term “mRNA” is used herein to indicate either the mature or processed messenger RNA, or the unprocessed nuclear pre-mRNA that encodes the human steroid 5α-reductase type 1 or the human steroid 5α-reductase type 2.

As used herein and unless otherwise indicated, the term “human steroid 5α-reductase type 1” or “human steroid 5α-reductase 1” is used herein to indicate one of the isozymes of human steroid 5α-reductase that is responsible for the reduction of androgens, such as testosterone, at the 5 position (Andersson, S, and Russell, D. W., 1990, Proc Natl Acad Sci USA, 87:3640-3644; Jenkins, E. P. et al., 1992, J. Clin. Inv., 89: 293-300). The activity of human steroid 5α-reductase type 1 on testosterone results in the enzymatic conversion of testosterone to 5α-reduced testosterone, or dihydrotestosterone, or DHT.

As used herein and unless otherwise indicated, the term “human steroid 5α-reductase type 2” or “human steroid 5α-reductase 2” is used herein to indicate one of the isozymes of human steroid 5α-reductase that is responsible for the reduction of androgens, such as testosterone, at the 5 position (Andersson, S, and Russell, D. W., 1990, Proc Natl Acad Sci USA 87:3640-3644; Jenkins, E. P. et al., 1992, J. Clin. Inv. 89: 293-300). The activity of human steroid 5α-reductase type 2 on testosterone results in the enzymatic conversion of testosterone to 5α-reduced testosterone, or dihydrotestosterone, or DHT.

As used herein and unless otherwise indicated, the term “reducing sebum secretion” is used herein to indicate the detectable reduction in the rate of sebum secretion which is effected by a composition of the invention. A composition of the invention may substantially reduce the rate of sebum secretion by about 10%, about 10-20%, about 20-30%, about 30-40%, about 40-50%, about 50-60%, about 60-70%, about 70-80% or more.

As used herein and unless otherwise indicated, the term “substantially inhibits expression” is used herein to indicate the detectable reduction in the level of expression of the encoded enzyme (translation) from a nucleic acid molecule which is effected by a composition of the invention. A composition of the invention may substantially inhibit the expression of human steroid 5α-reductase type 1 or type 2 by about 10%, about 10-20%, about 20-30%, about 30-40%, about 40-50% or more.

As used herein and unless otherwise indicated, the term “specifically binds” is used herein to refer to binding of nucleic acid molecules that occurs in an in vivo or in vitro cellular environment, so that a substantial inhibition of expression of the 5α-reductase type 1 or type 2 is exhibited without substantial effects attributed to binding to unrelated cellular nucleic acids.

As used herein and unless otherwise indicated, the term “expression” refers to the transcription and stable accumulation of sense (mRNA) or antisense RNA derived from the nucleic acid fragment of the invention. Expression may also refer to translation of mRNA into a polypeptide. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression of identical or substantially similar foreign or endogenous genes. (See, e.g., U.S. Pat. No. 5,231,020, incorporated herein by reference).

As used herein and unless otherwise indicated, the term “hybridize” or “hybridization” refers to the process by which a polynucleotide strand anneals with a complementary strand through base pairing under defined hybridization conditions. Specific hybridization is an indication that two nucleic acid sequences share a high degree of complementarity or reverse complementarity (i.e., wherein the polarity is reversed, for example, 5′->3′ is the reverse complement of a 3′->5′ oligonucleotide. Specific hybridization complexes form under permissive annealing conditions and remain hybridized after the “washing” step(s). The washing step(s) is particularly important in determining the stringency of the hybridization process, with more stringent conditions allowing less non-specific binding, (i.e., binding between pairs of nucleic acid strands that are not perfectly matched). Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 mu.g/ml sheared, denatured salmon sperm DNA. Generally, stringency of hybridization is expressed, in part, with reference to the temperature under which the wash step is carried out. Such wash temperatures are typically selected to be about 5° C. to 20° C. lower than the thermal melting point (Tm) for the specific sequence at a defined ionic strength and pH. The Tm is the temperature (under defined ionic strength and pH) at which 50% of the target sequence hybridizes to a perfectly matched probe. An equation for calculating Tm and conditions for nucleic acid hybridization are well known and can be found in Sambrook, J. et al. (1989) Molecular Cloning: A Laboratory Manual, 2.sup.nd ed., vol. 1-3, Cold Spring Harbor Press, Plainview N.Y.; specifically see volume 2, chapter 9.

As used herein, the term “vector” refers broadly to any plasmid or virus encoding an exogenous nucleic acid. The term is also be construed to include non-plasmid, non-phagemid and non-viral compounds which facilitate the transfer of nucleic acid into virions or cells, such as, for example, polylysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, a recombinant vaccinia virus, a recombinant adenovirus, a recombinant retrovirus, a recombinant adeno-associated virus, a recombinant avian pox virus, and the like (Cranage et al., 1986, EMBO J. 5:3057-3063; International Patent Application No. WO 94/17810, published Aug. 18, 1994; International Patent Application No. WO 94/23744, published Oct. 27, 1994). Examples of non-viral vectors include, but are not limited to, liposomes, polyamine derivatives of DNA, and the like.

### 6.2 General Description

The invention encompasses pharmaceutical compositions comprising a therapeutically or prophylactically effective amount of at least one anti-sense oligonucleotide, siRNA, or ribozyme or a pharmaceutically acceptable salt, solvate, hydrate, clathrate, polymorph, or prodrug thereof, which substantially inhibits the expression of human steroid 5α-reductase type 1 in a patient. In a particular embodiment, the antisense oligonucleotide is complementary to and specifically hybridizes with at least a portion of a nucleotide sequence that encodes the protein designated as human steroid 5α-reductase type 1. In another particular embodiment, the siRNA is complementary to and specifically hybridizes with at least a portion of a nucleotide sequence that encodes the protein designated as human steroid 5α-reductase type 1. In another particular embodiment, the ribozyme is complementary to and specifically hybridizes with at least a portion of a nucleotide sequence that encodes the protein designated as human steroid 5α-reductase type 1.

In another embodiment, the invention encompasses a pharmaceutical composition comprising a therapeutically effective amount of at least one anti-sense oligonucleotide, siRNA, or ribozyme or a pharmaceutically acceptable salt, solvate, hydrate, clathrate, polymorph, or prodrug thereof, which substantially inhibits the expression of human steroid 5α-reductase type 2 in a patient. In a particular embodiment, the antisense oligonucleotide is complementary to and specifically hybridizes with at least a portion of a nucleotide sequence that encodes the protein designated as human steroid 5α-reductase type 2. In another particular embodiment, the siRNA is complementary to and specifically hybridizes with at least a portion of a nucleotide sequence that encodes the protein designated as human steroid 5α-reductase type 2. In another particular embodiment, the ribozyme is complementary to and specifically hybridizes with at least a portion of a nucleotide sequence that encodes the protein designated as human steroid 5α-reductase type 2.

In another embodiment, the pharmaceutical composition is suitable for topical, intravenous, oral, or intranasal administration. In another embodiment, the pharmaceutical composition comprises a delivery formulation, which enhances the penetration of the anti-sense oligonucleotide across the stratum corneum of the skin. In a particular embodiment, the delivery formulation comprises ethyl alcohol, propylene glycol, glycerin, methyl cellulose, dimethyl isosorbide, polyethylene glycol ester, EDTA, pantethine and a divalent cation. In a more particular embodiment, the delivery formulation comprises about 15 to about 40% ethyl alcohol; about 0.5 to about 5.0% propylene glycol; about 0.5 to about 5.0% glycerin; about 0.1 to about 2.0% dimethyl isosorbide; about 0.1 to about 2.0% polyethylene glycol ester; about 0.01 to about 0.5% disodium EDTA; about 0.01 to about 0.2% pantethine and about 0.001 to about 2% divalent cation. In another embodiment, the pharmaceutical composition is formulated for topical administration for a period of at least four weeks. In another embodiment, the pharmaceutical composition is formulated for administration about once a day, about twice a day or up to about four times a day. In another embodiment, the delivery formulation may also be in the form of a polymer embedded with an oligonucleotide of the invention plasmid, for example, in the form of polymer microspheres including, but not limited to, poly(orthoester)microspheres.

In another embodiment, the pharmaceutical composition is useful in treating or preventing skin inflammation, disorders related to sebum secretion or excess sebum secretion, disorders related to sebum production or excess sebum production, steatoma, cystic acne, excess keratin production, comedones, papules, pustules, milia, seborrheic dermatitis, dandruff, seborrheic eczema, infantile seborrheic eczema, seborrheic keratosis, rosacea, perioral dermatitis, sebaceous cysts, acne vulgaris, oily skin, seborrheic wart, senile wart, basil cell papilloma, hirsutism, dermatosis paulosa nigra, benign prostatic hyperplasia, prostate cancer, urinary incontinence, androgenic alopecia, and male pattern baldness.

In another embodiment, the antisense oligonucleotide specifically hybridizes to an mRNA transcript that encodes the protein designated as human steroid 5α-reductase type 1. In another embodiment, the siRNA specifically hybridizes to an mRNA transcript that encodes the protein designated as human steroid 5α-reductase type 1. In another embodiment, the ribozyme specifically hybridizes to an mRNA transcript that encodes the protein designated as human steroid 5α-reductase type 1.

In another embodiment, the antisense oligonucleotide specifically hybridizes to a translation intitiation site, a 5′-untranslated sequences, 3′-untranslated sequences, any of the intron/exon junctions, or an intervening sequence of the mRNA transcript that is encoded by human steroid 5α-reductase type 1. In another embodiment, the siRNA specifically hybridizes to a translation intitiation site, a 5′-untranslated sequences, 3′-untranslated sequences, any of the intron/exon junctions, or an intervening sequence of the mRNA transcript that is encoded by human steroid 5α-reductase type 1. In another embodiment, the ribozyme specifically hybridizes to a translation intitiation site, a 5′-untranslated sequences, 3′-untranslated sequences, any of the intron/exon junctions, or an intervening sequence of the mRNA transcript that is encoded by human steroid 5α-reductase type 1.

In another embodiment, the antisense oligonucleotide specifically hybridizes to a 5′ cap site or a region adjacent to a 5′ cap site of the human steroid 5α-reductase type I transcript. In another embodiment, the siRNA specifically hybridizes to a 5′ cap site or a region adjacent to a 5′ cap site of the human steroid 5α-reductase type 1 transcript. In another embodiment, the ribozyme specifically hybridizes to a 5′ cap site or a region adjacent to a 5′ cap site of the human steroid 5α-reductase type 1 transcript.

In another embodiment, the antisense oligonucleotide specifically hybridizes to a portion of the coding sequence within the human steroid 5α-reductase type 1 mRNA transcript. In another embodiment, the siRNA specifically hybridizes to a portion of the coding sequence within the human steroid 5α-reductase type 1 mRNA transcript. In another embodiment, the ribozyme specifically hybridizes to a portion of the coding sequence within the human steroid 5α-reductase type 1 mRNA transcript.

In another embodiment, the antisense oligonucleotide is complementary to and specifically hybridizes with at least a portion of a nucleotide sequence that encodes the protein designated as human steroid 5α-reductase type 2. In another embodiment, the siRNA is complementary to and specifically hybridizes with at least a portion of a nucleotide sequence that encodes the protein designated as human steroid 5α-reductase type 2. In another embodiment, the ribozyme is complementary to and specifically hybridizes with at least a portion of a nucleotide sequence that encodes the protein designated as human steroid 5α-reductase type 2.

In another embodiment, the antisense oligonucleotide specifically hybridizes to an mRNA transcript that encodes the protein designated as human steroid 5α-reductase type 2. In another embodiment, the siRNA specifically hybridizes to an mRNA transcript that encodes the protein designated as human steroid 5α-reductase type 2. In another embodiment, the ribozyme specifically hybridizes to an mRNA transcript that encodes the protein designated as human steroid 5α-reductase type 2.

In another embodiment, the antisense oligonucleotide specifically hybridizes to a translation intitiation site, a 5′-untranslated sequences, 3′-untranslated sequences, any of the intron/exon junctions, or an intervening sequence of the mRNA transcript that is encoded by human steroid 5α-reductase type 2. In another embodiment, the siRNA specifically hybridizes to a translation intitiation site, a 5′-untranslated sequences, 3′-untranslated sequences, any of the intron/exon junctions, or an intervening sequence of the mRNA transcript that is encoded by human steroid 5α-reductase type 2. In another embodiment, the ribozyme specifically hybridizes to a translation intitiation site, a 5′-untranslated sequences, 3′-untranslated sequences, any of the intron/exon junctions, or an intervening sequence of the mRNA transcript that is encoded by human steroid 5α-reductase type 2.

In another embodiment, the antisense oligonucleotide specifically hybridizes to a 5′ cap site or a region adjacent to a 5′ cap site of the human steroid 5α-reductase type 2 transcript. In another embodiment, the siRNA specifically hybridizes to a 5′ cap site or a region adjacent to a 5′ cap site of the human steroid 5α-reductase type 2 transcript. In another embodiment, the ribozyme specifically hybridizes to a 5′ cap site or a region adjacent to a 5′ cap site of the human steroid 50α-reductase type 2 transcript.

In another embodiment, the antisense oligonucleotide specifically hybridizes to a portion of the coding sequence within the human steroid 5α-reductase type 2 mRNA transcript. In another embodiment, the siRNA specifically hybridizes to a portion of the coding sequence within the human steroid 5α-reductase type 2 mRNA transcript. In another embodiment, the ribozyme specifically hybridizes to a portion of the coding sequence within the human steroid 5α-reductase type 2 mRNA transcript.

In another embodiment, the anti-sense oligonucleotide comprises a sequence of at least 8 contiguous nucleotides selected from the group consisting of the complement of nucleotides 1-75 of SEQ ID NO: 1, the complement of nucleotides 620-682 of SEQ ID NO: 1 and the complement of nucleotides 1175-1250 of SEQ ID NO: 1.

In another embodiment, the anti-sense oligonucleotide comprises a sequence of at least 8 contiguous nucleotides selected from the group consisting of the complement of nucleotides 1-42 of SEQ ID NO: 28, the complement of nucleotides 10-30 of SEQ ID NO: 28 and the complement of nucleotides 21-230 of SEQ ID NO: 28.

In another embodiment, the anti-sense oligonucleotide comprises a sequence of at least 8 contiguous nucleotides selected from the group consisting of SEQ ID NOS: 2, 3, 4, 5, 6, 7, 8, 9, 10 and 11.

In another embodiment, the anti-sense oligonucleotide comprises a sequence of at least 8 contiguous nucleotides selected from the group consisting of SEQ ID NOS: 37, 38, 39, 40, 41, 42, 43, 44, 45, and 46.

In another embodiment, the pharmaceutical composition further comprises a second active agent, wherein the second active agent is an antifungal agent, an H¹ receptor antagonist, a retinoid, an anti-obesity drug, a hormone, a phosphodiesterase-5 inhibitor, an antibiotic, an anti-cancer agent, a topical steroid, or an astringent.

In another embodiment, the invention encompasses a method of treating or preventing a disorder that can be treated or prevented by inhibiting the conversion of testosterone to dihydrotestosterone, which comprises administering to a patient in need thereof a therapeutically effective amount of at least one anti-sense oligonucleotide, siRNA, or ribozyme or a pharmaceutically acceptable salt, solvate, hydrate, clathrate, polymorph, or prodrug thereof, which substantially inhibits the expression of human steroid 5α-reductase type 1 in a patient.

In another embodiment, the invention encompasses a method of treating or preventing skin inflammation, disorders related to sebum secretion or excess sebum secretion, disorders related to sebum production or excess sebum production, steatoma, cystic acne, excess keratin production, comedones, papules, pustules, milia, seborrheic dermatitis, dandruff, seborrheic eczema, infantile seborrheic eczema, seborrheic keratosis, rosacea, perioral dermatitis, sebaceous cysts, acne vulgaris, oily skin, seborrheic wart, senile wart, basil cell papilloma, hirsutism, dermatosis paulosa nigra, benign prostatic hyperplasia, prostate cancer, urinary incontinence, androgenic alopecia, and male pattern baldness in a patient in need thereof, which comprises administering to said patient a therapeutically effective amount of at least one anti-sense oligonucleotide, siRNA, or ribozyme or a pharmaceutically acceptable salt, solvate, hydrate, clathrate, polymorph, or prodrug thereof, which substantially inhibits the expression of human steroid 5α-reductase type 1 in a patient.

In another embodiment, the invention encompasses a method of treating or preventing a disorder that can be treated or prevented by inhibiting the conversion of testosterone to dihydrotestosterone, which comprises administering to a patient in need thereof a therapeutically effective amount of at least one anti-sense oligonucleotide, siRNA, or ribozyme or a pharmaceutically acceptable salt, solvate, hydrate, clathrate, polymorph, or prodrug thereof, which substantially inhibits the expression of human steroid 5α-reductase type 2 in a patient.

In another embodiment, the invention encompasses a method of treating or preventing skin inflammation, disorders related to sebum secretion or excess sebum secretion, disorders related to sebum production or excess sebum production, steatoma, cystic acne, excess keratin production, comedones, papules, pustules, milia, seborrheic dermatitis, dandruff, seborrheic eczema, infantile sebortheic eczema, seborrheic keratosis, rosacea, perioral dermatitis, sebaceous cysts, acne vulgaris, oily skin, seborrheic wart, senile wart, basil cell papilloma, hirsutism, dermatosis paulosa nigra, benign prostatic hyperplasia, prostate cancer, urinary incontinence, androgenic alopecia, and male pattern baldness in a patient in need thereof, which comprises administering to said patient a therapeutically effective amount of at least one anti-sense oligonucleotide, siRNA, or ribozyme or a pharmaceutically acceptable salt, solvate, hydrate, clathrate, polymorph, or prodrug thereof, which substantially inhibits the expression of human steroid 5α-reductase type 2 in a patient.

In another embodiment, the invention encompasses a dermatological composition for treating or preventing a disorder of the skin comprising at least one anti-sense oligonucleotide, siRNA, or ribozyme which substantially inhibits the expression of human steroid 5α-reductase type 1 or type 2 and further comprising an agent to enhance the penetration of the anti-sense oligonucleotide, siRNA. or ribozyme across the stratum corneum. In a particular embodiment, the agent to enhance the penetration is ethyl alcohol, propylene glycol, glycerin, dimethyl isosorbide, polyethylene glycol ester, EDTA, pantethine and a divalent cation. In a particular embodiment, the anti-sense oligonucleotide comprises a sequence of at least 8 contiguous nucleotides selected from the group consisting of the complement of nucleotides 1-75 of SEQ ID NO: 1, the complement of nucleotides 620-682 of SEQ ID NO: 1 and the complement of nucleotides 1175-1250 of SEQ ID NO: 1. In a particular embodiment, the anti-sense oligonucleotide comprises a sequence of at least 8 contiguous nucleotides selected from the group consisting of SEQ ID NOS: 2, 3, 4, 5, 6, 7, 8, 9, 10 and 11. In another particular embodiment, the anti-sense oligonucleotide comprises a sequence of at least 8 contiguous nucleotides selected from the group consisting of the complement of nucleotides 10-30 of SEQ ID NO: 28, the complement of nucleotides 620-682 of SEQ ID NO: 28 and the complement of nucleotides 1175-1250 of SEQ ID NO: 28. In a particular embodiment, the anti-sense oligonucleotide comprises a sequence of at least 8 contiguous nucleotides selected from the group consisting of SEQ ID NOS: 37, 38, 39, 40, 41, 42, 43, 44, 45, and 46.

In another embodiment, the dermatological composition further comprises a second agent, which inhibits the blockage of skin pores by sebaceous material. In a particular embodiment, the second molecule is retinoic acid, tretinoin, or retin-A.

### 6.3 Anti-Sense Oligonucleotides

In one embodiment, the oligonucleotides of the invention are anti-sense oligonucleotides. The anti-sense oligonucleotides of the invention may be prepared using standard synthetic methods known in the art and synthesized with a DNA synthesizer such as those available from Applied Biosystems, Inc., Beckman, Millipore, BioServe Biotechnologies Ltd., Biosset, Genemachines, etc. The oligonucleotides of the invention may also be chemically modified, as discussed below. Oligonucleotides of the invention can be constructed and purified by methods known in the art. The specific oligonucleotide sequences are constructed so as to have a nucleotide sequence that is complementary to a nucleotide sequence that comprises a portion of the gene that encodes human steroid 5α-reductase type 1 or human steroid 5α-reductase type 2. The oligonucleotides of the invention are typically 21 bases in length but may include as few as about 3 bases and as many as about 100 bases. The targeted sequences have been selected because they are essential for the translation of either the steroid 5α-reductase type 1 transcript or the steroid 5α-reductase type 2 transcript.

In an illustrative embodiment, the oligonucleotides of the invention comprise predetermined sequences of DNA ranging in size from about 8 bases to about 100 bases, which is sufficient to define a unique sequence in the human steroid 5α-reductase target type 1 transcript or human steroid 5α-reductase target type 2 transcript. Fewer than 8 bases may be used, however the degree of sequence specificity for the mRNA transcripts that encode human steroid 5α-reductase type 1 or human steroid 5α-reductase type 2 may decrease with decreasing lengths of the oligonucleotides. On the other hand, oligonucleotide sequences greater than about 100 bases may be subject to decreased uptake by cells. In one embodiment, the oligonucleotides comprise about 8 to about 50 bases. In another embodiment, the oligonucleotides comprise about 12 to about 20 bases. In still another embodiment, the oligonucleotides comprise about 15 to 25 bases.

The anti-sense oligonucleotides of the invention are intended for use in pharmaceutical compositions and achieve sufficient concentrations necessary to decrease the expression of a target mRNA or protein in a manner that provides therapeutic benefit. The oligonucleotides contemplated in this invention are constructed, or otherwise modified, so as to increase their stability by enhancing resistance to various degradative enzymes (e.g., nucleases). Such modifications function to permit the concentration of the oligonucleotide therapeutic to be maintained at a level that is sufficient so as to realize therapeutically effective benefit but cannot substantially alter the specificity of the oligonucleotide for its target sequence. Modifications that improve oligonucleotide stability or efficacy include, but are not limited to, modifications to the phosphate backbone, termini, sugar moieties and the individual nucleic acid bases. Conjugations to peptides, proteins, carbohydrates, lipids, vitamins or any other conjugation that increases therapeutic potency or efficacy can also be used. Also, any modifications resulting in stable secondary structures including circularization of the oligonucleotide and target sequence, and intrastrand joining of the 3′ to the 5′ termini through covalent bonds or hybridization and triple stranded binding to mRNA can also be made. Any modifications that reduce nuclease sensitivity while substantially maintaining the affinity and substrate specifically and solubility exhibited by unmodified oligonucleotides are within the scope of the invention. Other modifications to the oligonucleotides of the invention include, but are not limited to, DNA intercalators, photochemically activated cross-linking or cleaving agents. alkylating agents, and redox active nucleic acid cleaving groups. Vectors can be introduced to cells and produce long RNA molecules upon transcription. Such expression units for use in the invention will generally comprise the following elements, operably linked in a 5′ to 3′ orientation: a transcriptional promoter, a secretory signal sequence, a DNA sequence encoding the antisense oligonucleotide. Any arrangement of the antisense oligonucleotide may be used in the vectors of the invention. The selection of suitable promoters, signal sequences and terminators will be determined by the selected host cell and will be evident to one skilled in the art.

Several chemically modified oligonucleotides have been developed which substantially block or improve resistance to nuclease activity. These oligonucleotide modifications include phosphorothioate oligonucleotides, wherein one of the phosphate oxygens is replaced by sulfur. Another type of modification of oligonucleotides is accomplished by replacing the charged phosphate oxygen with a methyl group or other alkyl group. These nonionic DNA analogs include, for example, methyl phosphonates, alkyl-phosphorothioates, and O-alkyl phosphotriesters. An illustrative O-alkyl-phosphotriester of the invention is O-methylphosphotriester. Other DNA backbone modifications at the phosphate group include for example, phosphorodithioate, and phosphotriester oligonucleotides or oligonucleotides based on protein-nucleic acid structures or morpholino-like structures.

Various chemical modifications to either or both the 3′- or 5′-termini and the individual nucleic acid bases are known to improve stability of oligonucleotides to nucleases, stabilize the interaction of oligonucleotides with their specific target molecule, or enhance uptake of the oligonucleotides by cells. Moreover, chemical modifications to the 3′- or 5′-termini or modifications internal to the oligonucleotide can also be introduced as reporter molecules for example, to allow tracking of the oligonucleotide or as lipophilic moieties to enhance cell uptake. Such molecules can be introduced to both unmodified and backbone modified synthetic oligonucleotides. These moieties can be introduced for example, through thio or amino linkages to terminal hydroxyl or phosphate groups or to specific bases.

Other modifications to the oligonucleotides contemplated in this invention include for example, DNA intercalators, photochemically activated cross-linking or cleaving agents, alkylating agents and redox active nucleic acid cleaving groups.

Regardless of the modifications employed, the anti-sense oligonucleotides of the invention are designed to inhibit the expression of steroid 5α-reductase type 1 or steroid 5α-reductase type 2 and hybridize with sufficient specificity so as to reduce the potential of non-mechanistic-based toxicity. Investigations into the toxicity of other anti-sense oligonucleotides have not revealed significant damage or lethality to cells. To date, in vitro studies examining toxicity of anti-sense oligonucleotides have been limited primarily to modified oligomers wherein the phosphodiester linkages between the nucleosides have been replaced with either phosphorothioates or methylphosphonates. Under the conditions tested, exposure of a variety of cell lines to phosphorothioate oligomers has not resulted in any significant toxicity (Gao et al., 1990; Reed et al., 1990).

### 6.4 Ribozymes

Ribozymes, enzymatic RNA molecules, may also be used to catalyze the specific cleavage of RNA. The mechanism of action of the ribozyme of the invention involves sequence-specific hybridization of the ribozyme molecule to complementary target RNA, followed by endonucleolytic cleavage. In an illustrative embodiment, engineered hammerhead motif ribozymes of the invention may specifically and efficiently catalyze endonucleolytic cleavage of sequences encoding 5α-reductase type 1 or 5α-reductase type 2.

Specific ribozyme cleavage sites within any potential RNA target are initially identified by scanning the target molecule for ribozyme cleavage sites, including, but not limited to, the following sequences: GUA, GUU, and GUC. Once identified, short RNA sequences of, for example, between about 15 and about 20 ribonucleotides, corresponding to the region of the target gene containing the cleavage site, may be evaluated for secondary structural features, which may render the oligonucleotide inoperable. The suitability of candidate targets may also be evaluated by testing accessibility to hybridization with complementary oligonucleotides using ribonuclease protection assays.

Ribozymes of the invention may be prepared by any method known in the art for the synthesis of nucleic acid molecules. These include, but are not limited to, techniques for chemically synthesizing oligonucleotides such as solid phase phosphoramidite chemical synthesis. Alternatively, RNA molecules may be generated by in vitro and in vivo transcription of DNA sequences encoding 5α-reductase type 1 or 5α-reductase type 2. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

Illustrative hammerhead ribozyme designs for both Human 5α-reductase type I and type II are illustrated in Table 1. The ribozymes are listed in linear fashion reading in a 3′-->5′ manner (i.e., the reverse complement to the 5aR mRNA sequence).

TABLE 1

Identification of Sequence
having sequence identifiers

| | |
|---|---|
| SEO ID NO: | SEQUENCE AND COMMENTS |

| | |
|---|---|
| SEQ ID NO: 20 | 3′ ccccaaagcaggagugccugaguagucccg |
| | ccu-5′ for mRNA target sequence |
| | 153-163 of human 5α-reductase |
| | type 1 |
| SEQ ID NO: 21 | 3′ cggcacaagcaggagugccugaguaguccc |
| | cgac-5′ for mRNA target sequence |
| | 201-211 of human 5α-reductase |
| | type 1 |
| SEQ ID NO: 22 | 3′ ggacaaagcaggagugccugaguagucuug |
| | ccg-5′ for mRNA target sequence |
| | 229-239 of human 5α-reductase |
| | type 1 |
| SEQ ID NO: 23 | 3′ ggacaaagcaggagugccugaguagucuug |
| | ccg-5′ for mRNA target sequence |
| | 511-521 of human 5α-reductase |
| | type 1 |
| SEQ ID NO: 24 | 3′ guccaaagcaggagugccugaguagucagu |
| | cac-5′ for mRNA target sequence |
| | 31-41 of human 5α-reductase |
| | type 2 |
| SEQ ID NO: 25 | 3′ gaacaaagcaggagugccugaguagucugc |
| | agc-5′ for mRNA target sequence |
| | 99-109 of human 5α-reductase |
| | type 2 |
| SEQ ID NO: 26 | 3′ gaccaaagcaggagugccugaguagucacc |
| | acg-5′ for mRNA target sequence |
| | 183-193 of human 5α-reductase |
| | type 2 |
| SEQ ID NO: 27 | 3′ acccaaagcaggagugccugaguaguccca |
| | ugu-5′ for mRNA target sequence |
| | 441-451 of human 5α-reductase |
| | type 2 |

The ribozyme sequence -CAAAGCAGGAGUGCCUGAGUAGUC- (from nts 4-26) are conserved in all sequences. The first three bases and the last 6 are specific (i.e., reverse complements) for each targeted sequence in their respective mRNAs. The cleavage site is located in the target mRNA strand just after the CA located at position 4/5 of the ribozymes.

### 6.5 Small-Interfering RNA

The invention also encompasses compositions and methods comprising small interfering RNA (“siRNA”) for gene silencing. The siRNA of the invention can be prepared by several methods including, but not limited to, chemical synthesis, in vitro transcription, siRNA expression vectors, and PCR expression cassettes.

The siRNA of the invention are from about 10 to about 50, preferably from about 12 to about 40, more preferably from about 15 to about 30 and most preferably from about 19 to about 22 nucleotide (“nt”) double-stranded RNA.

Without being limited by theory it is believed that the siRNA of the invention works by cleaving and destroying its cognate RNA. The siRNA first assembles into RNA-induced silencing complexes, and it then activates the complex by unwinding its RNA strands. The unwound RNA strands subsequently guide the complex to the complementary RNA molecules, where the complex cleaves and destroys the cognate RNA, which results in RNA interference. “RNA interference (RNAi)” is the process of sequence-specific, posttranscriptional gene silencing initiated by siRNA. RNAi is seen in a number of organisms including, but not limited to, Drosophila, nematodes, fungi, plants, and humans, and is believed to be involved in anti-viral defense, modulation of transposon activity, and regulation of gene expression. During RNAi, siRNA induces degradation of target mRNA with consequent sequence-specific inhibition of gene expression.

As used herein, the terms “small interfering RNA,” “short interfering RNA,” or “siRNA” is a RNA duplex of nucleotides that is targeted to a gene interest. A “RNA duplex” refers to the structure formed by the complementary pairing between two regions of a RNA molecule. siRNA is “targeted” to a gene (e.g., the gene encoding for 5α-reductase) in that the nucleotide sequence of the duplex portion of the siRNA is complementary to a nucleotide sequence of the targeted gene or mRNA.

In one embodiment of the invention, the length of the duplex of siRNAs is about 30 nucleotides. In another embodiment, the duplex can be about 29, 28, 27, 26, 25, 24, 23, 22, 21, 20, 19, 18, 17, 16, 15, 14, 13, 12, 11 or 10 nucleotides in length. In another embodiment, the length of the duplex is about 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is about 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length.

In another embodiment, the siRNA is encoded by a nucleic acid sequence, and the nucleic acid sequence can also include a promoter. The nucleic acid sequence can also include a polyadenylation signal. In some embodiments, the polyadenylation signal is a synthetic minimal polyadelylation signal.

In another embodiment, the siRNA of the invention is introduced by transient transfection and effectively induces RNA interference in mammalian cultured cells in a sequence-specific manner. In a particular embodiment, the siRNAs result in greater than 80%, preferably greater than 90%, more preferably greater than 95%, and most preferably greater than 99% reduction in target RNA and protein levels. In another particular embodiment, the siRNAs of the invention are 21 nt dsRNAs with 2 nt 3′ overhangs. In a particular embodiment, sequence specificity of siRNA is stringent, as single base pair mismatches between the siRNA and its target mRNA dramatically reduce silencing. In another particular embodiment, sequence specificity of siRNA is non-stringent.

The siRNA can be used as small duplex RNA molecules or may be used in expression vectors to produce the double-stranded RNA molecules in vitro or in vivo. The siRNA molecules may be comprised of naturally occurring RNA molecules or may be comprised of modifications to the heterocycles, sugar, phosphate backbone, certain chemically attached pendent groups or can be altered in other manners such that the siRNA maintain their function as gene silencers in vitro and in vivo.

Illustrative siRNA inhibitors targeting human steroid 5α-reductase isoform Type 1 and Type II are listed in Table 2. The illustrative siRNA silence the expression of steroid 5α-reductase I and type II proteins and therefore can be used in vivo in humans to reduce the presence of the protein.

TABLE 2

Identification of Sequence
having sequence identifiers

| | |
|---|---|
| SEQ ID NO: | SEQUENCE AND COMMENTS |

| | |
|---|---|
| SEQ ID NO: 49 | 5′AAUCGUCAGACGAACUCAGUG-3′ |
| | 3′UUAGCAGUCUGCUUGAGUCAC-5′ |
| | (siRNA complement of NT |
| | 228-248 of human 5α- |
| | reductase type 1) |
| SEQ ID NO: 29 | 5′UCGUCAGACGAACUCAGUGUU |
| SEQ ID NO: 12 | 3′UUAGCAGUCUGCUUGAGUCAC |
| | (2-base U overhang or |
| | sticky ends on the 3′ end |
| | of the molecule) |
| SEQ ID NO: 50 | 5′AAUGGCGAAUUGAUGUUCUGUA-3′ |
| | 3′UUACCGCUUAACUACAAGACAU-5′ |
| | (siRNA complement of NT |
| | 491-511 of human 5α- |
| | reductase type 1) |
| SEQ ID NO: 30 | 5′UGGCGAAUUGAUGUUCUGUAUU |
| SEQ ID NO: 13 | 3′UUACCGCUUAACUACAAGACAU-5′ |
| | (2-base U overhang or |
| | sticky ends on the 3′ end |
| | of the molecule) |
| SEQ ID NO: 51 | 5′AAUGGCGAUUGAUGUUCUGUA-3′ |
| | 3′UUACCGCUAACUACAAGACAU-5′ |
| | (siRNA complement of NT |
| | 743-763 of human 5α- |
| | reductase type 1) |
| SEQ ID NO: 31 | 5′UGGCGAUUGAUGUUCUGUAUU-3′ |
| SEQ ID NO: 14 | 3′UUACCGCUAACUACAAGACAU-5′ |
| | (2-base U overhang or |
| | sticky ends on the 3′ end |
| | of the molecule) |
| SEQ ID NO: 52 | 5′AAUACGUAACUGCAGCCAACU-3′ |
| | 3′UUAUGCAUUGACGUCGGUUGA-5′ |
| | (siRNA complement of NT |
| | 712-732 of human 5α- |
| | reductase type 1) |
| SEQ ID NO: 32 | 5′UACGUAACUGGAGCCAACUUU-3′ |
| SEQ ID NO: 15 | 3′UUAUGCAUUGACGUCGGUUGA-5′ |
| | (2-base U overhang or |
| | sticky ends on the 3′ end |
| | of the molecule) |
| SEQ ID NO: 53 | 5′AAUCAGCUACAGGAUUCCACA-3′ |
| | 3′UUAGUCGAUGUCCUAAGGUGU-5′ |
| | (siRNA complement of NT |
| | 552-572 of human 5α- |
| | reductase type 2) |
| SEQ ID NO: 33 | 5′UGAGCUACAGGAUUCCACAUU-3′ |
| SEQ ID NO: 16 | 3′UUAGUCGAUGUCCUAAGGUGU-5′ |
| | (2-base U overhang or |
| | sticky ends on the 3′ end |
| | of the molecule) |
| SEQ ID NO: 54 | 5′AAGCACACGGAGAGCCUGAAG-3′ |
| | 3′UUCGUGUGCCUCUCGGACUUC-5′ |
| | (siRNA complement of NT |
| | 174-194 of human 5α- |
| | reductase type 2; |
| | Sequence location in |
| | Genbank Seq. ID. |
| | #NM_000348) |
| SEQ ID NO: 34 | 5′GCACACGGAGAGCCUGAAGUU-3′ |
| SEQ ID NO: 17 | 3′UUCGUGUGCCUCUCGGACUUC-5′ |
| | (2-base U overhang or |
| | sticky ends on the 3′ end |
| | of the molecule) |
| SEQ ID NO: 55 | 5′AAUGGAGUCCUUCAAGGCTAC-3′ |
| | 3′UUACCUCAGGAAGUUCCGAUG-5′ |
| | (siRNA complement of NT |
| | 391-411 of human 5α- |
| | reductase type 2; |
| | Sequence location in |
| | GenBank Seq. ID. |
| | #NM_000348) |
| SEQ ID NO: 35 | 5′UGGAGUCCUUCAAGGCTACUU-3′ |
| SEQ ID NO: 18 | 3′UUACCUCAGGAAGUUCGGAUG-5′ |
| | (2-base U overhang or |
| | sticky ends on the 3′ end |
| | of the molecule) |
| SEQ ID NO: 56 | 5′AAGCCUGGAGAAAUCAGCUAC-3′ |
| | 3′UUCGGACCUCUUUAGUCGAUG-5′ |
| | (siRNA complement of NT |
| | 541-561 of human 5α- |
| | reductase type 2; |
| | Sequence location in |
| | GenBank Seq. ID. |
| | #NM_000348) |
| SEQ ID NO: 36 | 5′GCCUGGAGAAAUCAGCUAGUU-3′ |
| SEQ ID NO: 19 | 3′UUCGGACCUCUUUAGUCGAUG-5′ |
| | (2-base U overhang or |
| | sticky ends on the 3′ end |
| | of the molecule) |

### 6.6 Inhibition of Human Steroid 5α-Reductase Type 1 and Type 2 by Anti-Sense Oligonucleotides, siRNA, and Ribozymes of the Invention

The invention encompasses antisense oligonucleotides, siRNA, and ribozymes that have a nucleotide sequence that is complementary to and capable of hybridizing with at least a portion of a nucleotide sequence that encodes 5α-reductase type 1 and 5α-reductase type 2 and capable of inhibiting the transcription of the gene or the translation of the mRNA transcript, thereby decreasing the concentration of the 5α-reductase type 1 or 5α-reductase type 2. The functions of RNA to be interfered with include, but are not limited to, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of human steroid 5α-reductase 1 or human steroid 5α-reductase 2. In particular, oligonucleotides, siRNA, and ribozymes are provided, which have a base sequence capable of hybridizing to the mRNA transcript of human steroid 5α-reductase type 1 and human steroid 5α-reductase 2 when administered alone or in combination with other agents (e.g., finasteride) that decrease steroid 5α-reductase activity or substantially inhibit the expression of other steroid 5α-reductase genes. Hybridization of the oligonucleotides to steroid 5α-reductase type 1 mRNA or 5α-reductase type 2 mRNA substantially blocks the translation of the mRNA transcript. Without being limited by theory it is believed that because the enzyme steroid 5α-reductase is essential for the conversion of testosterone (T) to dihydrotestosterone (DHT), the level of DHT will decrease in androgen-responsive tissues resulting in therapeutic benefit to patients exhibiting conditions that are characterized by an over production of DHT.

The oligonucleotides, siRNA, and ribozymes of the invention have been selected because they are capable of hybridizing with a high degree of specificity to regions of the transcript including the translation initiation site, along with sequences 5′ or 3′ to the translation initiation site. Other oligonucleotides, siRNA, and ribozymes have been selected that hybridize to the 5′ cap region of the mRNA or sequences 3′ or 5′ to the cap site. Additional oligonucleotide sequences of the invention are complementary to sequences found in the 3′ untranslated region of the steroid 5α-reductase type 1 gene and are unique to the steroid 5α-reductase type 1 gene. Such sequences are capable of hybridizing with specificity to sequences found in the 3′-untranslated region of the steroid 5α-reductase type 1 mRNA transcript. In addition to the sequences described above, other sequences contained within the 5α-reductase type 1 transcript are targeted. Moreover, the instant invention further contemplates anti-sense oligonucleotides complementary to any portion of the steroid 5α-reductase type 1 gene and which are capable of cross-linking DNA, intercalating DNA or binding more tightly by mechanisms such as, for example, triple stranding.

Furthermore, additional oligonucleotides, siRNA, and ribozyme sequences of the invention are complementary to sequences found in the 3′ untranslated region of the steroid 5α-reductase type 2 gene and are unique to the steroid 5α-reductase type 2 gene. Anti-sense oligonucleotide, siRNA, and ribozyme sequences of the invention were also developed that are complementary to sequences found in the 3′ untranslated region of the steroid 5α-reductase type 2 gene and are unique to the steroid 5α-reductase type 2 gene. Such sequences are capable of hybridizing with specificity to sequences found in the 3′-untranslated region of the steroid 5α-reductase type 2 mRNA transcript. In addition to the sequences described above, other sequences contained within the 5α-reductase type 2 transcript are targeted. Moreover, the instant invention further contemplates anti-sense oligonucleotides, siRNA, and ribozymes complementary to any portion of the steroid 5α-reductase type 2 gene and which are capable of cross-linking DNA, intercalating DNA, or binding more tightly by mechanisms such as, for example, triple stranding.

Thus, the invention contemplates that anti-sense oligonucleotides, siRNA, and ribozymes are capable of substantially inhibiting the expression of the steroid 5α-reductase type 1 and 5α-reductase type 2.

In order for anti-sense oligonucleotides, siRNA, or ribozymes to become successful therapeutics the oligonucleotides or modified oligonucleotides must be taken up by the cell that expresses the target gene, pre-mRNA, or mRNA or be expressed within the cell. The anti-sense oligonucleotides, siRNA, and ribozymes of the invention are constructed so as to insure that the oligonucleotide will pass through the plasma membrane and achieve an intracellular concentration that is sufficient to substantially decrease the expression of steroid 5α-reductase type 1 or steroid 5α-reductase type 1. The anti-sense oligonucleotides, siRNA, and ribozymes of the invention that are constructed to bind to the steroid 5α-reductase type 1 or 5α-reductase type 2 gene or mRNA can be further modified, if necessary, to enable them to pass through the nuclear membrane in levels that are sufficient to reduce transcription. Recent attempts at enhancing the cellular uptake of anti-sense oligonucleotides have employed a wide variety of techniques including the use of lipoproteins, (de Schmidt et al., 1991), and a wide variety of conjugates, such as poly-L-lysine and cholesterol (Goodchild, 1990). Conjugation of cholesterol to the 5′ end of an oligonucleotide has been reported to result in a molecule that exhibited reduced serum clearance due to reduction in renal excretion, compared to that observed with control oligo-deoxynucleotides (ODNs) (de Schmidt et al., 1991). As a result, the conjugation of cholesterol to ODNs may allow an increase in the delivery of drug to liver cells via the LDL transport mechanism. Liposomes containing anti-sense oligonucleotides can also be targeted to specific cell types by the addition of cell-specific antibodies (Leonetti et al., 1990; Mizuno et al., 1990). These and other methods of achieving and maintaining adequate intracellular concentrations of the oligonucleotides, including making prodrugs as defined herein, are contemplated by this invention and include other methods and compositions that have the capacity to enhance cellular uptake or decrease the efflux of internalized oligonucleotides. Such modifications do not alter the specificity of the oligonucleotide for its target sequence.

### 6.7 Therapeutic Uses of Compositions of the Invention

In accordance with the invention, the oligonucleotides, siRNA or ribozymes or a pharmaceutically acceptable salt, solvate, hydrate, clathrate, polymorph, or prodrug thereof are useful for administration to a patient, preferably a human, with or at risk of skin inflammation, disorders related to sebum secretion or excess sebum secretion, disorders related to sebum production or excess sebum production, steatoma, cystic acne, excess keratin production, comedones, papules, pustules, milia, seborrheic dermatitis, dandruff, seborrheic eczema, infantile seborrheic eczema, seborrheic keratosis, rosacea, perioral dermatitis, sebaceous cysts, acne vulgaris, oily skin, seborrheic wart, senile wart, basil cell papilloma, hirsutism, dermatosis paulosa nigra, benign prostatic hyperplasia, prostate cancer, urinary incontinence, androgenic alopecia, and male pattern baldness. In one embodiment, “treatment” or “treating” refers to an amelioration of a disease or disorder, or at least one discernible symptom thereof. In another embodiment, “treatment” or “treating” refers to delaying the onset of a disease or disorder or inhibiting the progression thereof, either physically (e.g., stabilization of a discernible symptom), physiologically (e.g., stabilization of a physical parameter), or both.

In certain embodiments, the compounds of the invention or the compositions of the invention are administered to a patient, preferably a human, as a preventative measure against such disorders or diseases. As used herein, “prevention” or “preventing” refers to a reduction of the risk of acquiring a given disease or disorder. In one embodiment, the compositions of the invention are administered as a preventative measure to a patient, preferably a human having a genetic predisposition to skin inflammation, disorders related to sebum secretion or excess sebum secretion, disorders related to sebum production or excess sebum production, steatoma, cystic acne, excess keratin production, comedones, papules, pustules, milia, seborrheic dermatitis, dandruff, seborrheic eczema, infantile seborrheic eczema, seborrheic keratosis, rosacea, perioral dermatitis, sebaceous cysts, acne vulgaris, oily skin, seborrheic wart, senile wart, basil cell papilloma, hirsutism, dermatosis paulosa nigra, benign prostatic hyperplasia, prostate cancer, urinary incontinence, androgenic alopecia, and male pattern baldness.

In another embodiment, the compositions of the invention are administered as a preventative measure to a patient having a non-genetic predisposition to a skin inflammation, disorders related to sebum secretion or excess sebum secretion, disorders related to sebum production or excess sebum production, steatoma, cystic acne, excess keratin production, comedones, papules, pustules, milia, seborrheic dermatitis, dandruff, seborrheic eczema, infantile seborrheic eczema, seborrheic keratosis, rosacea, perioral dermatitis, sebaceous cysts, acne vulgaris, oily skin, seborrheic wart, senile wart, basil cell papilloma, hirsutism, dermatosis paulosa nigra, benign prostatic hyperplasia, prostate cancer, urinary incontinence, androgenic alopecia, and male pattern baldness.

6.7.1 Treatment or Prevention of Skin Inflammation

The invention provides methods for the treatment or prevention of a skin inflammation comprising administering to a patient a therapeutically or prophylactically effective amount of an antisense oligonucleotide, siRNA, or ribozyme of the invention or a composition comprising an antisense oligonucleotide, siRNA, or ribozyme of the invention and a pharmaceutically acceptable vehicle. As used herein, the term “skin inflammation” includes, but is not limited to, epidermal edema, characterized clinically by vesicles, poorly marginated redness, edema, oozing, crusting, scaling, usually pruritus, and lichenification caused by scratching or rubbing.

6.7.2 Treatment or Prevention of Steatoma

The invention provides methods for the treatment or prevention of a steatoma comprising administering to a patient a therapeutically or prophylactically effective amount of an antisense oligonucleotide, siRNA, or ribozyme of the invention or a composition comprising an antisense oligonucleotide, siRNA, or ribozyme of the invention and a pharmaceutically acceptable vehicle. As used herein, the term “steatoma” includes, but is not limited to, a cyst containing matter like suet or a slow-growing benign cyst containing follicular and keratinous material frequently found on the scalp, ears, face, back, or scrotum.

6.7.3 Treatment or Prevention of Cystic Acne

The invention provides methods for the treatment or prevention of cystic acne comprising administering to a patient a therapeutically or prophylactically effective amount of an antisense oligonucleotide, siRNA, or ribozyme of the invention or a composition comprising an antisense oligonucleotide, siRNA, or ribozyme of the invention and a pharmaceutically acceptable vehicle. As used herein, the term “cystic acne” refers to a form of acne which results from the bacterial infection of cysts deep within the skin. Without treatment cystic acne may result in scarring.

6.7.4 Treatment or Prevention of Comedones

The invention provides methods for the treatment or prevention of comedones comprising administering to a patient a therapeutically or prophylactically effective amount of an antisense oligonucleotide, siRNA, or ribozyme of the invention or a composition comprising an antisense oligonucleotide, siRNA, or ribozyme of the invention and a pharmaceutically acceptable vehicle. As used herein, the term “comedones” refers an acne lesion including, but not limited to, open, noninflammatory comedo (e.g., a blackhead) or closed comedo (e.g., a whitehead).

6.7.5 Treatment or Prevention of Papule

The invention provides methods for the treatment or prevention of papule comprising administering to a patient a therapeutically or prophylactically effective amount of an antisense oligonucleotide, siRNA, or ribozyme of the invention or a composition comprising an antisense oligonucleotide, siRNA, or ribozyme of the invention and a pharmaceutically acceptable vehicle. As used herein, the term “papule” refers to a small inflamed elevation of skin that is nonsuppurative.

6.7.6 Treatment or Prevention of Milia

The invention provides methods for the treatment or prevention of milia comprising administering to a patient a therapeutically or prophylactically effective amount of an antisense oligonucleotide, siRNA, or ribozyme of the invention or a composition comprising an antisense oligonucleotide, siRNA, or ribozyme of the invention and a pharmaceutically acceptable vehicle. As used herein, the term “milia” refers to little plugs of keratin in the glands of the skin of the face. The resulting bumps are a common feature of newborns' faces. The tiny bumps of milia are no larger than a millimeter or two. They are most common on the tip of the nose or chin, and are frequently seen on the cheeks and forehead. Less commonly, they will be found on the upper trunk or limbs and even on the penis.

6.7.7 Treatment or Prevention of Seborrheic Dermatitis

The invention provides methods for the treatment or prevention of seborrheic dermatitis comprising administering to a patient a therapeutically or prophylactically effective amount of an antisense oligonucleotide, siRNA, or ribozyme of the invention or a composition comprising an antisense oligonucleotide, siRNA, or ribozyme of the invention and a pharmaceutically acceptable vehicle. As used herein, the term “seborrheic dermatitis” refers to a red, scaly, itchy rash most commonly seen on the scalp, sides of the nose, eyebrows, eyelids, skin behind the ears, and middle of the chest. Other areas, such as the navel (belly button), buttocks, skin folds under the arms, axillary regions, breasts, and groin, may also be involved. This condition is most common in three age groups—infancy when it's called “cradle cap,” middle age, and the elderly. Cradle cap usually clears without treatment by age 8 to 12 months. In some infants, seborrheic dermatitis may develop only in the diaper area where it could be confused with other forms of diaper rash. When seborrheic dermatitis develops at other ages it can come and go. Seborrheic dermatitis may be seasonally aggravated particularly in northern climates; it is common in people with oily skin or hair, and may be seen with acne or psoriasis. A yeast-like organism may be involved in causing seborrheic dermatitis.

Seborrheic dermatitis may occur in patients with diseases of the nervous system, such as Parkinson's disease. Patients recovering from stressful medical conditions, such as a heart attack, may also develop this problem. People in hospitals or nursing homes and those with immune system disorders appear to be more prone to this disorder as well. Thus, drugs useful in treating these disorders can be combined with the compositions of the present invention can be used.

6.7.8 Treatment or Prevention of Seborrheic Eczema

The invention provides methods for the treatment or prevention of seborrheic eczema comprising administering to a patient a therapeutically or prophylactically effective amount of an antisense oligonucleotide, siRNA, or ribozyme of the invention or a composition comprising an antisense oligonucleotide, siRNA, or ribozyme of the invention and a pharmaceutically acceptable vehicle. As used herein, the term “seborrheic eczema” refers to eczema of the scalp that can affect adults as well as infants. Both conditions generally start on the scalp, but have the ability to spread elsewhere on the body. Seborrheic eczema in infants under 1 year is also known as cradle cap. It appears on the scalp as crusty, yellowish or reddish skin. It does look unappealing on such cute beings, but it does not seem to cause soreness or itchiness to infants. This condition is thought to be associated with a deficiency of bictin in the affected infants. It goes away after a few months but can be helped by using bath oils and moisturizing creams. Adults between the ages of 20 and 40 can be affected by this type of eczema. Seborrheic eczema occurs on the scalp as a mild case of dandruff. The condition turns red, inflamed, and flaky and can quickly spread down to the face, ears, neck, and chest. This condition is known to be intensified by stress and is thought to be related to a blockage of sebaceous glands.

6.7.9 Treatment or Prevention of Seborrheic Keratosis

The invention provides methods for the treatment or prevention of seborrheic keratosis comprising administering to a patient a therapeutically or prophylactically effective amount of an antisense oligonucleotide, siRNA, or ribozyme of the invention or a composition comprising an antisense oligonucleotide, siRNA, or ribozyme of the invention and a pharmaceutically acceptable vehicle. As used herein, the term “seborrheic keratosis” refers to non-cancerous growths of the outer layer of skin. There may be just one growth, or many which occur in clusters. They are usually brown, but can vary in color from light tan to black. They vary in size from a fraction of an inch in diameter to larger than a half-dollar. A main feature of seborrheic keratoses is their waxy, “pasted-on” or “stuck-on” look. They sometimes look like a dab of warm brown candle wax that has dropped onto the skin. Seborrheic keratoses are most often found on the chest or back, although, they can also be found on the scalp, face, neck, or almost anywhere on the body. They appear less often below the waist. Since they are not caused by sunlight, they can be found on sun-exposed or covered areas. When they first appear, the growths usually begin one at a time as small, rough, itchy bumps. Eventually, they thicken and develop a rough, warty surface.

6.7.10 Treatment or Prevention of Rosacea.

The invention provides methods for the treatment or prevention of rosacea comprising administering to a patient a therapeutically or prophylactically effective amount of an antisense oligonucleotide, siRNA, or ribozyme of the invention or a composition comprising an antisense oligonucleotide, siRNA, or ribozyme of the invention and a pharmaceutically acceptable vehicle. As used herein, the term “rosacea” refers to a skin disease that causes redness and swelling on the face. Often referred to as “adult acne,” rosacea may begin as a tendency to flush or blush easily, and progress to persistent redness in the center of the face that may gradually involve the cheeks, forehead, chin, and nose. It also may involve the ears, chest and back. As the disease progresses, small blood vessels and tiny pimples begin to appear on and around the reddened area; however, unlike acne, there are no blackheads. Pimples of rosacea appear on the face as small, red bumps, some of which may contain pus. These may be accompanied by the development of many tiny blood vessels on the surface of the skin and persistent redness of the face. In more advanced cases of rosacea, a condition called rhinophyma (ryno-fi-ma) may develop. The oil glands enlarge causing a bulbous, enlarged red nose and puffy cheeks. Thick bumps can develop on the lower half of the nose and nearby cheeks. Rhinophyma occurs less commonly in women.

6.7.11 Treatment or Prevention of Perioral Dermatitis

The invention provides methods for the treatment or prevention of perioral dermatitis comprising administering to a patient a therapeutically or prophylactically effective amount of an antisense oligonucleotide, siRNA, or ribozyme of the invention or a composition comprising an anti-sense oligonucleotide, siRNA, or ribozyme of the invention and a pharmaceutically acceptable vehicle. As used herein, the term “perioral dermatitis” refers to redness of the skin with small red bumps or even pus bumps and mild peeling in the area around the mouth. Sometimes the bumps are the most obvious feature, and the disease can look a lot like acne. The areas most affected are within the borders of the lines from the nose to the sides of the lips, and the chin. There is frequent sparing of a small band of skin that borders the lips. Occasionally, the areas around the nose, eyes, and cheeks can be affected. Sometimes there is mild itching and/or burning.

6.7.12 Treatment or Prevention of Acne Vulgaris

The invention provides methods for the treatment or prevention of acne vulgaris comprising administering to a patient a therapeutically or prophylactically effective amount of an antisense oligonucleotide, siRNA, or ribozyme of the invention or a composition comprising an antisense oligonucleotide, siRNA, or ribozyme of the invention and a pharmaceutically acceptable vehicle. As used herein, the term “acne vulgaris” refers to an inflammatory condition of the sebaceous glands of the skin. It consists of red, elevated areas on the skin that may develop into pustules and even further into cysts that can cause scarring. Acne vulgaris occurs mostly on the face, neck, and back of most commonly teenagers and to a lesser extent of young adults. The condition results in part from excessive stimulation of the skin by androgens (male hormones). Bacterial infection of the skin also appears to play a role.

6.7.13 Treatment or Prevention of Sebaceous Cysts

The invention provides methods for the treatment or prevention of sebaceous cysts comprising administering to a patient a therapeutically or prophylactically effective amount of an antisense oligonucleotide, siRNA, or ribozyme of the invention or a composition comprising an antisense oligonucleotide, siRNA, or ribozyme of the invention and a pharmaceutically acceptable vehicle. As used herein, the term “sebaceous cysts” refers to a closed sac occurring just under the skin which contains a “pasty” or “cheesy” looking substance. A foul odor is also often present in the substance, which fills sebaceous cysts. Keratin is a protein that creates the sac of cells called sebaceous cysts. These small lumps or bumps can occur just under the skin of the vagina, genitalia, breast, abdomen, face, neck, or elsewhere on the body.

6.7.14 Treatment or Prevention of Basil Cell Papilloma

The invention provides methods for the treatment or prevention of basil cell papilloma comprising administering to a patient a therapeutically or prophylactically effective amount of an antisense oligonucleotide, siRNA, or ribozyme of the invention or a composition comprising an antisense oligonucleotide, siRNA, or ribozyme of the invention and a pharmaceutically acceptable vehicle. As used herein, the term “basil cell papilloma” refers to wart like growths commonly seen as small lumps on the eyelid margin or eyelid skin.

6.7.15 Treatment or Prevention of Meibomian Cyst

The invention provides methods for the treatment or prevention of meibomian cysts comprising administering to a patient a therapeutically or prophylactically effective amount of an antisense oligonucleotide or ribozyme, siRNA, of the invention or a composition comprising an antisense oligonucleotide, siRNA, or ribozyme of the invention and a pharmaceutically acceptable vehicle. As used herein, the term “meibomian cyst” refers to a lump in the oil glands of the eyelid resulting in a visible lump on the eyelid surface. These can become infected resulted in pain and swelling within the eyelid.

6.7.16 Treatment or Prevention of Hirsutism

The invention provides methods for the treatment or prevention of hirsutism comprising administering to a patient a therapeutically or prophylactically effective amount of an antisense oligonucleotide, siRNA, or ribozyme of the invention or a composition comprising an antisense oligonucleotide, siRNA, or ribozyme of the invention and a pharmaceutically acceptable vehicle. As used herein, the term “hirsutism” refers to increased hair growth in women. It refers to a male pattern of hair (i.e., in the moustache and beard areas) or occurring more thickly than usual on the limbs. There may be hairs on the chest or an extension of pubic hair on to the abdomen and thighs. Hirsutism is nearly always genetic in origin.

6.7.17 Treatment or Prevention of Dermatosis Papulosa Nigra

The invention provides methods for the treatment or prevention of dermatosis papulosa nigra comprising administering to a patient a therapeutically or prophylactically effective amount of an antisense oligonucleotide, siRNA, or ribozyme of the invention or a composition comprising an antisense oligonucleotide, siRNA, or ribozyme of the invention and a pharmaceutically acceptable vehicle. As used herein, the term “dermatosis papulosa nigra” refers to a benign cutaneous condition that is usually characterized by multiple, small, hyperpigmented, asymptomatic papules on the face.

6.7.18 Treatment or Prevention of Benign Prostatic Hyperplasia

The invention provides methods for the treatment or prevention of benign prostatic hyperplasia comprising administering to a patient a therapeutically or prophylactically effective amount of an antisense oligonucleotide, siRNA, or ribozyme of the invention or a composition comprising an antisense oligonucleotide or ribozyme of the invention and a pharmaceutically acceptable vehicle. As used herein, the term “benign prostatic hyperplasia” refers to a benign neoplasm (non cancerous enlargement of the prostate gland) in men, and has a high prevalence that increases with age. The increase in size of the prostate inside its capsule exerts pressure on the urethra, which passes through the capsule, resulting in obstruction to urine flow.

6.7.19 Treatment or Prevention of Prostate Cancer

The invention provides methods for the treatment or prevention of prostate cancer comprising administering to a patient a therapeutically or prophylactically effective amount of an antisense oligonucleotide, siRNA, or ribozyme of the invention or a composition comprising an antisense oligonucleotide, siRNA, or ribozyme of the invention and a pharmaceutically acceptable vehicle. As used herein, the term “prostate cancer” refers to the growth of cancer cells in the prostate. Like that of normal prostate cells, it is stimulated by male hormones, especially testosterone.

6.7.20 Treatment or Prevention of Androgenic Alopecia

The invention provides methods for the treatment or prevention of androgenic alopecia comprising administering to a patient a therapeutically or prophylactically effective amount of an antisense oligonucleotide, siRNA, or ribozyme of the invention or a composition comprising an antisense oligonucleotide, siRNA, or ribozyme of the invention and a pharmaceutically acceptable vehicle. As used herein, the term “androgenic alopecia” refers to male and female pattern baldness. In androgenic alopecia the hair loss occurs slowly over years. It can start anytime after age twenty. There is usually a family history of hair loss. In women, the hair slowly becomes thin throughout the scalp and bald spots usually do not occur. Men first develop hair loss at the temples, followed by an enlarging bald spot on top of the head. Androgenic alopecia is thought to be due to the hair growing tissue's sensitivity to hormones. Androgenic alopecia in men is also referred to male pattern baldness.

6.7.21 Treatment or Prevention of Urinary Incontinence

The invention provides methods for the treatment or prevention of urinary incontinence comprising administering to a patient a therapeutically or prophylactically effective amount of an antisense oligonucleotide, siRNA, or ribozyme of the invention or a composition comprising an antisense oligonucleotide, siRNA, or ribozyme of the invention and a pharmaceutically acceptable vehicle. As used herein, the term “urinary incontinence” refers to unintentional loss of urine. Most cases of urinary incontinence fall under one of the following six major subtypes: stress incontinence, overactive bladder, mixed incontinence, overflow incontinence, lack of continuity or deformity, or functional incontinence.

### 6.8 Therapeutic/Prophylactic Administration and Compositions

The anti-sense oligonucleotides, siRNA, and ribozymes of the invention are administered to achieve efficacious levels in target tissues. Thus, the anti-sense oligonucleotides, siRNA, and ribozymes of the invention may be administered by any number of routes, including, but not limited to, topical, dermal, subdermal, transdermal, parenteral, oral, rectal, or by other means including surgical implantation of an oligonucleotide or ribozyme containing pump or other slow release formulation. The compositions are usually employed in the form of pharmaceutical compositions having a nucleotide sequence at least substantially complementary to a portion of the mRNA transcript of the human steroid 5α-reductase type 1 gene or human steroid 5α-reductase type 2 gene along with a suitable pharmaceutical carrier.

Due to the activity of the compounds and compositions of the invention, they are useful in veterinary and human medicine. As described above, the compositions of the invention are useful for the treatment or prevention of skin inflammation, disorders related to sebum secretion or excess sebum secretion, disorders related to sebum production or excess sebum production, steatoma, cystic acne, excess keratin production, comedones, papules, pustules, milia, seborrheic dermatitis, dandruff, seborrheic eczema, infantile seborrheic eczema, seborrheic keratosis, rosacea, perioral dermatitis, sebaceous cysts, acne vulgaris, oily skin, seborrheic wart, senile wart, basil cell papilloma, hirsutism, dermatosis paulosa nigra, benign prostatic hyperplasia, prostate cancer, urinary incontinence, androgenic alopecia, and male pattern baldness.

The invention provides methods of treatment and prophylaxis by administration to a patient of a therapeutically effective amount of a composition comprising an oligonucleotide, siRNA, or ribozyme of the invention. The patient is an animal, including, but not limited, to an animal such a cow, horse, sheep, pig, chicken, turkey, quail, cat, dog, mouse, rat, rabbit, guinea pig, etc., and is more preferably a mammal, and most preferably a human.

The compositions of the invention may be administered by any convenient route, for example, orally, topically, by intravenous infusion or bolus injection, by absorption through epithelial or mucocutaneous linings (e.g., oral mucosa, rectal and intestinal mucosa, etc.) and may be administered together with another biologically active agent. The compositions of the invention, are preferably administered topically. Administration can be systemic or local. Various delivery systems are known, e.g., encapsulation in liposomes, microparticles, microcapsules, capsules, etc., and can be used to administer a composition of the invention. In certain embodiments, more than one composition of the invention is administered to a patient. Methods of administration include, but are not limited to, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, scalp, or skin. The preferred mode of administration is left to the discretion of the practitioner, and will depend in-part upon the site of the medical condition. In most instances, administration will result in the release of the composition of the invention for maximum uptake by a cell.

In specific embodiments, it may be desirable to administer one or more compositions of the invention locally to the area in need of treatment. This may be achieved, for example, and not by way of limitation, by topical application (e.g. as a cream); by local infusion during surgery (e.g., in conjunction with a wound dressing after surgery); by injection; by means of a catheter; by means of a suppository; or by means of an implant, said implant being of a porous, non-porous, or gelatinous material, including membranes, such as sialastic membranes, or fibers. In one embodiment, administration can be by direct injection at the site (or former site) of an atherosclerotic plaque tissue.

In certain embodiments, for example, for the treatment of skin inflammation, androgenic alopecia, acne vulgaris, it may be desirable to introduce one or more compounds of the invention directly to the skin or scalp by any suitable route, including topically, intrathecal or epidural injection. The compositions of the invention may be applied not only to oily skin or skin affected by acne, but also prophylactically to skin to prevent acne and the build-up of sebum that leads to acne, other blemishes and local inflammations. Those who would particularly benefit from treatment with the compositions of the invention are teenagers, young adults, and women in certain phases of the menstrual cycle.

In another embodiment, the composition is prepared in a form suitable for administration directly or indirectly to surface areas of the body for direct application to affected areas. This formulation includes, but is not limited to, anti-drying agents (e.g., pantethine), penetration enhancers (e.g., dimethyl isosorbide), accelerants (e.g., isopropylmyristate) or other common additives that are known in the industry aad used for topical applications (e.g., glycerin, propylene glycol, polyethylene glycols, ethyl alcohol, liposomes, lipids, oils, creams, or emollients). In addition, the delivery vehicles of the invention may include compounds that have a beneficial effect on skin pores, such as retinoic acid (i.e., Retin-A), which removes sebum plugs from pores; antioxidants (e.g., butylated hydroxyanisole); or chelating preservatives (e.g., disodium EDTA). Further, the delivery vehicles of the invention may be adjusted to take into account the area of the body's skin that is being treated and/or the size or nature (i.e., single or double stranded) of the nucleic acid being used for topical applications.

To further improve the effectiveness of topical formulations, which deliver the compositions across the stratum corneum, phosphorothioate oligonucleotides may be used. Phosphorothioate modified oligonucleotides have shown considerable promise for use in anti-sense applications. Phosphorothioate-modified oligonucleotides are used since these modifications are known to exhibit significant improvement in the biological half-life of the oligonucleotides when compared to unmodified oligonucleotides. Typically, phosphorothioate-modified oligonucleotides exhibit the same characteristics of naturally occurring DNA molecules. Both natural and phosphorothioate-based DNA oligonucleotides of the same length are approximately the same size, form the same secondary and tertiary structures and possess a large net negative charge with one negative charge at each inter-nucleoside linkage. However, phosphorothioate-modified oligonucleotides have greater resistance to nucleolytic degradation because of the presence of a sulfur atom that is substituted for one of the non-bridging oxygen atoms of the phosphodiester inter-nucleoside linkages.

While there has been some work on delivering DNA across the stratum corneum, there are other approaches that might have applicability. The major constraint appears to be the size of the molecule. While a number of formulations have demonstrated the capacity to carry compounds across the stratum corneum, these compounds are not as large as a DNA molecule. For example, for an anti-sense oligomer comprised of 20 nucleotides, the anticipated molecular weight would be about 6,200 daltons. The resistance of the stratum corneum however, may be modulated in such a way by specific formulations, that delivery of naked DNA oligonucleotides, ribozymes, aptamers, or even cDNA constructs may be achievable.

Most drugs are not able to cross the stratum corneum. However, enhanced penetration can be achieved using a class of compounds known collectively as “penetration enhancers.” Alcohols, sulphoxides, fatty acids, esters, Azone, pyrrolidones, urea and polyoles are just some of the members of this class of compounds (Kalbitz et al., 1996). The objectives of these penetration enhancers are to change the solubility and diffusivity of the drug in the stratum corneum, thus some modulate their effects through the lipid pathway while others modify diffusion via the polar pathway.

Addition of various concentrations of the enhancer glycerin have been shown to enhance the penetration of cyclosporin (Nakashima et al., 1996). The use of terpene-based penetration enhancers with aqueous propylene glycol have also shown the capacity to enhance topical delivery rates of 5-fluorouricil (Yamane et al., 1995). 5-fluorouricil, 5-FU, is a model compound for examining the characteristics of hydrophilic compounds in skin permeation studies. Thus, the addition of terpenes in polylene glycol (up to 80%) were able to enhance the flux rate into skin.

Dimethyl isosorbide (DMI) is another penetration enhancer that has shown promise for pharmaceutical formulations. DMI is a water-miscible liquid with a relatively low viscosity (Zia et al., 1991). DMI undergoes complexation with water and polylene glycol but not polyethylene glycol. It is the ability for DMI to complex with water that provides the vehicle with the capacity to enhance the penetration of various steroids. Maximum effects were seen at a DMI:water ratio of 1:2. Evidence in the literature suggests that the effect of pH on DMI is an important consideration when using DMI in various formulations (Brisaert et al., 1996).

Pulmonary administration can also be employed, (e.g., by use of an inhaler or nebulizer), and formulation with an aerosolizing agent, or via perfusion in a fluorocarbon or synthetic pulmonary surfactant. In certain embodiments, the compounds of the invention can be formulated as a suppository, with traditional binders and vehicles such as triglycerides.

In another embodiment the compositions of the invention can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the compositions of the invention can be delivered in a controlled release system. In one embodiment, a pump may be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target area to be treated, (e.g., the liver), thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

The present compositions will contain a therapeutically effective amount of a composition of the invention, optionally more than one composition of the invention, preferably in purified form, together with a suitable amount of a pharmaceutically acceptable vehicle so as to provide the form for proper administration to the patient.

The term “vehicle” refers to a diluent, adjuvant, excipient, or carrier with which a composition of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compositions of the invention and pharmaceutically acceptable vehicles are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents. The preferred range of concentrations for the above composition of an effective delivery vehicle for nucleic acid-based compounds are as follows: ethyl alcohol 15-40%; propylene glycol 0.5-5.0%; glycerin 0.5-5.0%; dimethyl isosorbide 0.1-2.0%; polyethylene glycol ester (as Laureth-4) 0.1-2.0%; disodium EDTA 0.01-0.5%; pantethine 0.01-0.2%, divalent cation (copper, magnesium, manganese, zinc, copper litnium, etc.) 0.01-2% and water to 100%.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in “Remington's Pharmaceutical Sciences” by E. W. Martin. In an illustrative embodiment, the compositions of the invention are formulated in accordance with routine procedures as a pharmaceutical composition adapted for intravenous administration to human beings. Typically, compositions of the invention for intravenous administration are solutions in sterile isotonic aqueous buffer. Where necessary, the compositions may also include a solubilizing agent. Compositions for intravenous administration may optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition of the invention is to be administered by intravenous infusion, it can be dispensed, for example, with an infusion bottle containing sterile pharmaceutical grade water or saline. Where the compound of the invention is administered by injection, an ampoule of sterile water for injection or saline can be provided so that the ingredients may be mixed prior to administration.

Compositions of the invention for oral delivery may be in the form of tablets, lozenges, aqueous or oily suspensions, granules, powders, emulsions, capsules, syrups, or elixirs. Compounds and compositions of the invention for oral delivery can also be formulated in foods and food mixes. Orally administered compositions may contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compositions of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

The amount of a composition of the invention that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. However, suitable dosage ranges for oral administration are generally about 0.01 nanoMolar (“nM”) to 200 millimolar (“mM”) of a antisense oligonucleotide, siRNA, or ribozyme of the invention per kilogram body weight. In specific preferred embodiments of the invention, the oral dose is 0.01 nm to 70 mM per kilogram body weight, more preferably 0.1 nM to 50 mM per kilogram body weight, more preferably 0.5 nM to 20 mM per kilogram body weight, and yet more preferably 1 nM to 10 mM per kilogram body weight. In a most preferred embodiment, the oral dose is 5 nM of a composition of the invention per kilogram body weight. The dosage amounts described herein refer to total amounts administered; that is, if more than one composition of the invention is administered, the preferred dosages correspond to the total amount of the compounds of the invention administered. Oral compositions preferably contain 10% to 95% active ingredient by weight.

Suitable dosage ranges for intravenous (i.v.) administration are 0.01 nM to 100 mM per kilogram body weight, 0.1 nM to 35 mM per kilogram body weight, and 1 nM to 10 mM per kilogram body weight. Suitable dosage ranges for intranasal administration are generally about 0.01 nM/kg body weight to 1 nM/kg body weight. Suppositories generally contain 0.01 nM to 50 mM of a composition of the invention per kilogram body weight and comprise active ingredient in the range of 0.5% to 10% by weight. Recommended dosages for intradermal, intramuscular, intraperitoneal, subcutaneous, epidural, sublingual, intracerebral, intravaginal, transdermal administration or administration by inhalation are in the range of 0.001 nM to 200 mM per kilogram of body weight. Suitable doses of the compounds of the invention for topical administration are in the range of 0.001 nM to 1 mM, depending on the area to which the compound is administered. Effective doses may be extrapolated from dose-response curves derived from in vitro or animal model test systems. Such animal models and systems are well known in the art.

In the case of parenteral administration (e.g., for the treatment of, for example, benign prostatic hyperplasia), the compositions of the invention may be encapsulated in a liposome “envelope” that is coupled to an antibody directed against human prostate-specific proteins so as to provide target cell selectivity. The specific nature of the formulation is determined by the desired route of administration, e.g. topical, parenteral, oral, rectal, surgical implantation or by other means of local (intraprostatic) delivery. The dosage is determined for the route of administration. The amount of oligonucleotide or ribozyme in the composition can range from about 0.01 to 99% by weight of the composition. Direct treatment of the prostate may involve the perineal administration of a suitable preparation of at least one anti-sense oligonucleotide under echographic control. The injection may be made in either the zone of hyperplasia or in the external gland. A similar approach has been reported for the treatment of chronic prostatitis through the intraprostatic injection of antibiotics (Jimenez et al., 1988). In these studies transitory post-injection hemospermia together with pain during or after injection were the sole adverse effects observed with this therapy.

Compositions for rectal administration are prepared with any of the usual pharmaceutical excipients, including for example, binders, lubricants and disintegrating agents. The composition may also include cell penetration enhancers, such as aliphatic sulfoxides. In a preferred embodiment, the composition of the present invention is in the form of a suppository.

The invention also provides pharmaceutical packs or kits comprising one or more containers filled with one or more compounds of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains more than one compound of the invention. In another embodiment, the kit comprises a compound of the invention and another lipid-mediating compound, including but not limited to, retin-A, aloe, anti-oxidants, essential Oils, glycerin, chamomile, glycolic acid, calendula, apricot, avocado, ascorbic acid, an anti-cancer agent, benzoyl peroxide, alpha blockers, anti-androgens, non-peptide hormones; antibiotics, etc.

The compounds of the invention are preferably assayed in vitro and in vivo, for the desired therapeutic or prophylactic activity, prior to use in humans. For example, in vitro assays can be used to determine whether administration of a specific composition of the invention or a combination of compositions of the invention is preferred for inhibiting levels of 5α-reductase. The compositions of the invention may also be demonstrated to be effective and safe using animal model systems.

Other methods will be known to the skilled artisan and are within the scope of the invention.

### 6.9 Combination Therapy

In certain embodiments of the present invention, the compositions of the invention can be used in combination therapy with at least one other therapeutic agent. The compositions of the invention and the therapeutic agent can act additively or, more preferably, synergistically. In a preferred embodiment, a composition comprising an anti-sense oligonucleotide or a ribozyme of the invention is administered concurrently with the administration of another therapeutic agent, which can be part of the same composition as the anti-sense oligonucleotide or a ribozyme of the invention or a different composition. In another embodiment, a composition comprising an anti-sense oligonucleotide or a ribozyme of the invention is administered prior or subsequent to administration of another therapeutic agent. As many of the disorders for which the compositions of the invention are useful in treating are chronic disorders, in one embodiment combination therapy involves alternating between administering a a composition comprising an anti-sense oligonucleotide or a ribozyme of the invention and a composition comprising another therapeutic agent (e.g., to minimize the toxicity associated with a particular drug). The duration of administration of each drug or therapeutic agent can be, for example, one month, three months, six months, or a year. In certain embodiments, when a composition of the invention is administered concurrently with another therapeutic agent that potentially produces adverse side effects including but not limited to toxicity, the therapeutic agent can advantageously be administered at a dose that falls below the threshold at which the adverse side is elicited.

The present compositions can be administered together with an antifungal agent. Antigungals for use in combination with the compositions of the invention include, but are not limited to, ampho B, clotrimazole, fluconazole, flucytosine, Itraconazole, ketoconazole, nystatin, terbinafine, butenafine, ciclopirox, enconazole, naftifine, miconazole, oxiconazole, and tolnaftate.

The present compositions can also be administered together with an H¹ receptor antagonist. H¹ receptor antagonists for use in combination with the compositions of the invention include, but are not limited to, diphenynhydramine, pyrilamine, promethazine, chlorpheniramine, chlorcyclizine, loratadine, descarboethoxyloratidine, pheniramine, tripelenamine, and cyclizine.

The present compositions can also be administered together with a compound from the vitamin A family of retinoids. Retinoids for use in combination with the compositions of the invention include, but are not limited to, retinol, all-trans-retinol, all-trans-14-hydroxyretroretinol, all-trans-retinal, all trans-retinoic acid, all-trans-3,4-didehydroretinoic acid, 9-cis-retinoic acid, 11-cis-retinal, 13-cis-retinal, and 13-cis-retinoic acid.

The present compositions can also be administered together with an anti-obesity drug. Anti-obesity drugs for use in combination with the compounds of the invention include, but are not limited to, beta-adrenergic receptor agonists, preferably beta-3 receptor agonists, fenfluramine, dexfenfluramine, sibutramine, bupropion, fluoxetine, and phentermine.

The present compositions can also be administered together with a hormone. Hormones for use in combination with the compounds of the invention include but are not limited to thyroid hormone, estrogen and insulin. Preferred insulins include but are not limited to injectable insulin, transdermal insulin, inhaled insulin, or any combination thereof. As an alternative to insulin, an insulin derivative, secretagogue, sensitizer or mimetic may be used. Insulin secretagogues for use in combination with the compounds of the invention include but are not limited to forskolin, dibutryl cAMP or isobutylmethylxanthine (IBMX).

The present compositions can also be administered together with a phosphodiesterase-5 inhibitor. Phosphodiesterase-5 inhibitor drugs for use in combination with the compounds of the invention include, but are not limited to, sildenafil, vardenafil, and tadalafil.

The present compositions can also be administered together with an antibiotic agent. Antibiotic drugs for use in combination with the compounds of the invention include, but are not limited to, aminoglycosides, streptomycin, neomycin, anamycin, amikacin, gentamicin, tobramycin, streptomycin B, dihydrostreptomycin, spectinomycin, penicillin, ampicillin, hetacillin, amoxicillin, carbenicillin, cephalosporins, cephaloridine, cephalothin sodium, cephaloglycin dihydratem, cephalexin monohydrate, tetracycline, tetracycline hydrochloride, oxytetracycline hydrochloride, chlorotetracycline hydrochloride, doxocycline monohydrate, methacydine hydrochloride, 7-chloro-6-dimethyltetracycline, erythamycin, sulfonamides, carbomycin, oleanodmycin, troleandomycin, polymixin B collistin, and chloramphenicol.

The present compositions can also be administered together with an anti-cancer agent. Anti-cancer drugs for use in combination with the compounds of the invention include, but are not limited to, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel.

The present compositions can also be administered together with a topical steroids. Topical steroids for use in combination with the compounds of the invention include, but are not limited to, corticosteroids, such as, methylprednisolone and prednisone, and modified steroids such as, but not limited to, 3-phenanthridinones and 17-N,N-diethylcarbamoyl-4-methyl-4-aza-5-androstan-3-one (i.e., 4-MA).

The present compositions can also be administered together with an astringent. Astringents for use in combination with the compounds of the invention include, but are not limited to, witch hazel, aluminum acetate, aluminum sulfate, zinc oxide, zinc acetate, sodium bicarbonate, and calamine.

The present compositions can also be administered together with a second agent which inhibits the enzymatic activity of HS5αR type 1. Because such an agent inhibits the enzymatic conversion of testosterone to dihydrotestosterone (DHT), its inclusion in the composition provides a second mechanism for reducing local concentrations of DHT. The anti-sense oligonucleotide serves to inhibit further HS5αR production at the level of mRNA, while the enzyme inhibitor serves to inhibit the activity of remaining HS5αR enzyme.

The present compositions can also be administered together with a second agent that inhibits formation of the sebaceous material that plugs pores in the skin and creates an environment in which acne bacteria thrive. One exemplary agent is Retin-A (retinoic acid, tretinoin), which is believed to function by preventing the proliferation of sebocytes in sebaceous glands. As sebocytes mature, they accumulate sebum and move towards the sebaceous duct, where they rupture, releasing sebum and cellular debris into the pilosebaceous canal. These waste materials then form plugs (Downing et al., 1982). Once the pores are unblocked, sebum flows freely. Combining these two agents yields a composition that keeps skin pores open and reduces sebum flow, thereby altering skin conditions so that they are less favorable to bacterial growth and infection. Because the bacteria in the pores can convert sebum to free fatty acids, which trigger inflammation, reducing levels of P. acnes results in reduced acne and inflammation.

The present compositions can also be administered together with an agent that promotes hair regrowth including, but not limited to, minoxodil.

Without further description, it is believed that one of ordinary skill in the art can, using the preceding description and the following illustrative examples, make and utilize the compounds of the present invention and practice the claimed methods. The following working examples therefore, specifically point out preferred embodiments of the present invention, and are not to be construed as limiting in any way the remainder of the disclosure.

### 7. ILLUSTRATIVE TARGET GENES AND TARGET SEQUENCES

According to a one embodiment, the invention is directed to methods of treating or preventing disorders using oligonucleotides, siRNA, and ribozymes of the invention, which interfere with the expression of the enzyme 5α-reductase type 1 and type 2, or with the expression of the androgen receptor itself. Suitable oligonucleotides include antisense oligonucleotides, siRNA, ribozymes, third strand oligonucleotides, and triplex oligonucleotide pairs. It is preferred that suitable oligonucleotides, siRNA, and ribozymes are antisense oligonucleotides, siRNA, or ribozymes.

According to an illustrative embodiment of the invention, there are provided methods of treating or preventing skin inflammation, disorders related to excess sebum secretion, steatoma, cystic acne, excess keratin production, comedones, papule, milia, seborrheic dermatitis, seborrheic eczema, infantile seborrheic eczema, seborrheic keratosis, rosacea, perioral dermatitis, sebaceous cysts, acne vulgaris, oily skin, seborrheic wart, senile wart, basil cell papilloma, hirsutism, dermatosis paulosa nigra, benign prostatic hyperplasia, prostate cancer, urinary incontinence, androgenic alopecia, and male pattern baldness by preventing or interfering with the expression of the human 5-alpha-reductase enzyme by administration of an oligonucleotide or ribozyme of the invention, which is complementary to a target sequence on the DNA or an mRNA transcribed therefrom, which codes for the human 5-alpha-reductase or to a sequence immediately upstream from the transcription start site for the mRNA. The oligonucleotide administered may be an anti-sense oligonucleotide, a third strand oligomer, or a triplex oligomer pair. The antisense oligomer is complementary to a sequence of RNA transcribed from a target gene, to a single-stranded DNA target sequence, or to a single RNA or DNA strand contained within a duplex. The third strand oligonucleotide has a base sequence selected so that it is capable of hydrogen bonding with a sequence of a double stranded nucleic acid and forming a triple helix complex therewith. The first and second oligomers of the triplex oligomer pair have sequences selected such that they are complementary to and capable of hydrogen bonding with a targeted single-stranded nucleic acid sequence of a target gene or its transcription product or to a single strand of a duplex and together with the single stranded nucleic acid form a triple helix complex.

The target gene is selected from those genes encoding the enzyme 5-alpha-reductase type 1 and type 2 and is considered to include a target sequence immediately upstream from the transcription start site of that gene. Preferably the target sequence would include sequences from −500 to +20 (relative to the transcription start site) of the 5-alpha reductase gene. More preferably suitable sequences would include target sequences in the area from −100 to +20 (relative to the transcription start site) of the 5-alpha reductase gene.

Oligomers of appropriate length, preferably from about 8 to 100 nucleotides, more preferably from about 12 to about 40 nucleotides, and most prefereably from about 15 to about 25 nucleotides are selected so as to be adjacent to or cover these sites when hybridized to the target, in part or in whole. Preferred sites when the target sequence is mRNA include, in 5-alpha-reductase genes, the 5′ untranslated region, the translation initiation region including regions slightly downstream of the AUG start codon (preferably up to about 20 nucleotides downstream from the AUG initiation codon), splice acceptors, splice donors, and the 3′ untranslated region.

Thus, according to a preferred aspect of the invention, antisense oligonucleotides of the invention of the appropriate length, preferably from about 8 to 40 nucleosides and more preferably from about 12 to about 23 nucleosides especially from about 15 to about 25 nucleosides, are selected so as to have sequences which hybridize sites immediately adjacent to these sites or hybridize with and cover these sites, in part or wholly, as defined by the nucleotide positions included above for 5-alpha-reductase and the androgen receptor.

When antisense oligonucleotides are used, the sequence of the oligonucleotides is the reverse complement of the sequence of the targeted region so as to be able to hybridize to the targeted region.

When third strand oligonucleotides are used, the oligonucleotides are selected to form sequence-specific hydrogen bonding interactions with the double stranded nucleic acid target.

When triplex oligomer pairs are used, the first and second oligomers are selected so as to form sequence specific hydrogen bonding interactions with a single stranded nucleic acid, and together form a triple helix structure.

To assist in understanding the present invention, the following examples are included which describe the results of a series of experiments. The following examples relating to this invention should not, of course, be construed in specifically limiting the invention and such variations of the invention, now known or later developed, which would be within the purview of one skilled in the art are considered to fall within the scope of the present invention as hereinafter claimed.

### 8. ILLUSTRATIVE EXAMPLES

### 8.1 Example 1

### Preparation Of Anti-Sense Oligonucleotides

A series of oligonucleotides whose base sequences are substantially complementary to specific nucleotide sequences contained in the human steroid 5α-reductase type 1 and type 2 mRNA transcripts (SEQ ID NO: 1 and SEQ ID NO. 28, respectively) are prepared as follows. Anti-sense oligonucleotides are synthesized on an automated, solid phase DNA synthesizer using standard techniques practiced in the art. The corresponding phosphorothioate oligodeoxyribonucleotides are synthesized using standard procedures (Iyer et al., 1990). The sequences of the oligonucleotides, which are designed to inhibit human steroid 5α-reductase type 1 biosynthesis, are listed in Table 3, and the sequences of the oligonucleotides, which are designed to inhibit human steroid 5α-reductase type 2 biosynthesis, are listed in Table 4. The oligonucleotides of the invention may also be prepared by solution phase synthesis or by the reverse transcriptase technique. The oligonucleotides can also be prepared, for example, by genetic engineering techniques or other synthetic methods.

TABLE 3
Anti-Sense Oligonucleotides Which Target
Steroid 5α-Reductase Type 1

| DPC NO | SEQ. ID NO. | TARGET REGION |
|---|---|---|
| DPC 1524 | 2 | 5′-UNTRANSLATED (21-1) |
| DPC 1525 | 3 | 5′-UNTRANSLATED (27-7) |
| DPC 1526 | 4 | AUG CODON (36-16) |
| DPC 1528 | 5 | AUG CODON (48-28) |
| DPC 1527 | 6 | AUG CODON (43-23) |
| DPC 1529 | 7 | CODING (64-44) |
| DPC 1530 | 8 | CODING (678-658) |
| DPC 1531 | 9 | 3′-UNTRANSLATED (858-838) |
| DPC 1532 | 10 | 3′-UNTRANSLATED (986-966) |
| DPC 1533 | 11 | 3′-UNTRANSLATED(1240-1220) |

TABLE 4
Anti-Sense Oligonucleotides Which Target
Steroid 5α-Reductase Type 2

| DPC NO | SEQ. ID NO. | TARGET REGION |
|---|---|---|
| DPC 1674 | 37 | 5′-UNTRANSLATED (1-21) |
| DPC 1675 | 38 | 5′-UNTRANSLATED (6-26) |
| DPC 1676 | 39 | AUG CODON (10-30) |
| DPC 1677 | 40 | AUG CODON (13-33) |
| DPC 1678 | 41 | AUG CODON (22-42) |
| DPC 1679 | 42 | CODING (210-230) |
| DPC 1680 | 43 | CODING (1275-1295) |
| DPC 1681 | 44 | 3′-UNTRANSLATED (1680-1700) |
| DPC 1682 | 45 | 3′-UNTRANSLATED (1860-1880) |
| DPC 1683 | 46 | 3′-UNTRANSLATED (961-981) |

### 8.2 Example 2

### Effect of Treatment with Anti-Sense Oligonucleotides

To determine the effects of an anti-sense oligonucleotide on the expression of steroid 5α-reductase type 1, enzyme activity is measured using either cultured cells that normally express the enzyme, such as the human genital skin fibroblast cell line Hs68 (CRL 1635, American Type Culture Collection, Manassas, Va.) or cultured human prostate cells or cell lines that were transfected with the desired steroid 5α-reductase cDNA. Appropriate cells for this purpose include, but are not limited to, simian COS cells and human embryonal kidney 293 cells (CRL 1573). Expression plasmids containing the human steroid 5α-reductase type 1 cDNA have been described previously (Andersson and Russell, 1990). Simian COS cells (CRL 1651) are grown and transfected using a DEAE dextran protocol (Esser et al., 1988) The cells are maintained at 37° C. in an atmosphere of 95% O₂, 5% CO₂ and propagated according to methods previously described (Jenldns et al., 1992; Thigpen and Russell, 1992).

In cells that constitutively express steroid 5α-reductase, oligonucleotides are added to the medium at a concentration of 0.001 to 10.0 μM. In the transient transfection assay, the oligonucleotides are either co-transfected with the steroid 5α-reductase cDNA or added to the medium at a concentration of 0.001 to 10.0 μM following transfection. Cells are plated in multi-well tissue culture plates. The size of the well used for a particular assay is determined by the level of steroid 5α-reductase expressed by a given cell line.

The substrate is prepared by dissolving unlabeled testosterone (Sigma Chemical Co., St. Louis, Mo.) in absolute ethanol followed by the addition of either [7⁻³H(N)]-testosterone (23.3 Ci/mmole) or [¹⁴C]-testosterone (50 mCi/mmol) (New England Nuclear Boston, Mass.). The solvent is evaporated under a stream of nitrogen and the steroids reconstituted in an appropriate medium.

The medium in the sample wells is aspirated and replaced with fresh medium containing the radiolabeled substrate. An additional three wells containing medium and substrate but no cells are also included in order to account for the non-enzymatic metabolism of the substrate. The plates are then returned to the incubator and incubated for a length of time dependent on the level of steroid 5α-reductase expressed by the cell line.

At the end of the incubation period, the medium is collected and transferred to an extraction tube containing 5 ml of toluene-ethanol (9:1), to which has been added 40-250 μg each of unlabeled carrier steroids (estriol, estradiol, estrone, 5α-androstan-3α,17β-diol, 5α-androstan-3β,17β-diol, 4-androstene-3,17-dione, 5α-androstan-3,17β-dione, testosterone, and 5α-dihydrotestosterone (Steraloids, Inc. Wilton, N.H.). Depending upon the method used to detect the radiolabeled steroids the extraction solvent may or may not contain 1,000 and 10,000 dpm of [4-¹⁴C]-dihydrotestosterone (steroid 50-60 mCi/mmol) and [4-¹⁴C]-testosterone (50 mCi/mmol) (New England Nuclear Boston, Mass.); respectively. In assays that employ [7-³H(N)]-testosterone as a substrate, the [¹⁴C]-steroids are included as recovery standards to quantify procedural losses. A small amount of NaCl is also added to the extraction tubes to prevent foaming. The samples are vortexed for approximately 30 seconds and then centrifuged for 10 minutes at 500×g. The organic phase is collected and the solvent evaporated. The steroids are then reconstituted in dichloromethane-methanol (9:1) and analyzed by thin layer chromatography.

The extracted samples are applied to silica gel 60F₂₅₄, 0.25 mm thick, thin layer chromatography plates (EM Science, Cincinnati, Ohio). The plates are developed in a solvent system of chloroform:ethyl acetate (3:1, Mallinckrodt Inc. Paris, Ky.). The plates are allowed to developed until the solvent front migrates to within 2.0 cm of the top of the plate. After removal from the tanks, the plates are air dried. The plates are then viewed under 254 nm UV light and the visible spots marked. The plates are then sprayed with primulin (0.001% in acetone-water (4:1) according to the method of Wright (Wright, 1971) which allows the identification of additional steroids under 365 nm UV light. The spots are scraped from the plate using a glass wool plugged Pasteur pipette attached to a vacuum line. The steroids are eluted directly into scintillation vials by the addition of 0.2 ml of dichloromethane followed by two washes of 2.0 ml of methanol. The organic solvent is evaporated, and 10.0 ml of scintillation fluid (Ready Organic, Beckman Instruments, Inc. Fullerton Calif.) are added. Samples are analyzed by liquid scintillation spectrometry. In assays that employ [¹⁴C]-testosterone as the substrate, steroid metabolism is analyzed directly using the Phosphorimager imaging system (Molecular Dynamics, Inc., San Jose, Calif.).

Following removal of the media for extraction, the cells are washed with phosphate buffered saline (PBS, pH 7.4), and then harvested by exposure to a trypsin-EDTA solution (0.025% trypsin, 0.265 mM EDTA). The cells are collected and centrifuged at 1400×g for 5 minutes. The supernatant is decanted and the cells resuspended in PBS. An aliquot of the cell suspension is counted in a Coulter Counter Model ZM (Coulter Electronics, Ltd., Luton Beds, England). The remaining cells are sonicated and the protein determined according to the method of Bradford (Bradford, 1976). Corrections are made for procedural losses, and the data expressed as percent inhibition based on steroid concentration in terms of picomoles per mg protein or picomoles/10⁵ cells.

### 8.3 Example 3

### Treatment of Subjects with an Anti-Sense Oligonucleotide to Assess Effects on Sebum Secretion

Human subjects were treated topically twice a day for 4 weeks and their level of sebum production was measured. The basal level of sebum production was determined by using SebuTape (which quantitates the amount of sebum produced over a short period of time), and the subjects then swabbed their foreheads with a cotton swab containing either the vehicle or the vehicle plus the anti-sense inhibitor (SEQ ID NO: 5) at a concentration of 10 μM. Five subjects received vehicle alone and five received a composition containing the anti-sense oligonucleotide in the same vehicle. To ensure that the anti-sense inhibition was reproducible, the study was repeated. The results showed that in both studies, the anti-sense compounds were effective, yielding about 37% and about 27% reductions compared to base-line sebum production after 4 weeks.

### 8.4 Example 4

### Treatment of Subjects with an Anti-Sense Oligonucleotide to Assess Effects on Sebum Secretion

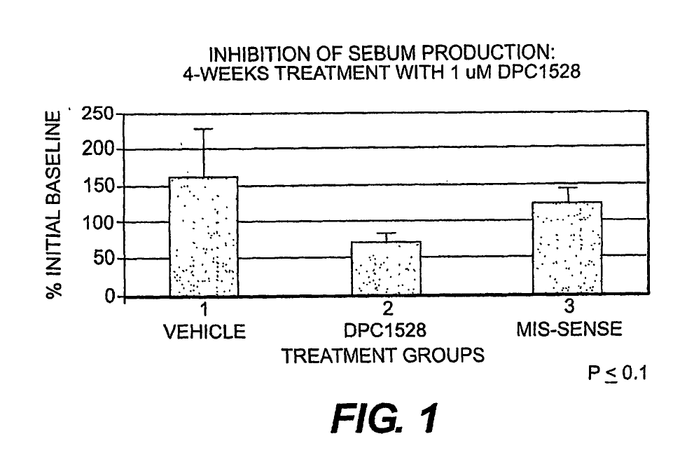

Three groups of five subjects each were treated with a topical formulation containing an anti-human steroid 5α-reductase type 1 (anti-HS5αR) anti-sense oligonucleotide (1 μM DPC1528), or a mis-sense or scrambled anti-sense oligonucleotide, or a control formulation for four weeks. Prior to treatment, each subject's rate of sebum secretion was monitored using a SebuMeter SM810 (Courage & Khazaka Electronics). For SebuMeter readings, the subjects were situated in an environmentally controlled room (temperature and humidity) for 30 minutes. Their foreheads were first swabbed to remove existing sebum, and the amount of new sebum secreted was measured. Subjects applied about 0.5 ml of one of the three solutions twice daily to their foreheads for a period of 4 weeks, and their sebum secretion rate was then remeasured by SebuMeter. DPC1528 produced a 25% decrease in sebum production while the mis-sense control with the scrambled sequence did not alter sebum production (see FIG. 1). Significance was determined using a TTest (two tailed), with an n=5.

### 8.5 Example 5

### Treatment of Subjects with an Anti-Sense Oligonucleotide to Assess Effects on Sebum Secretion

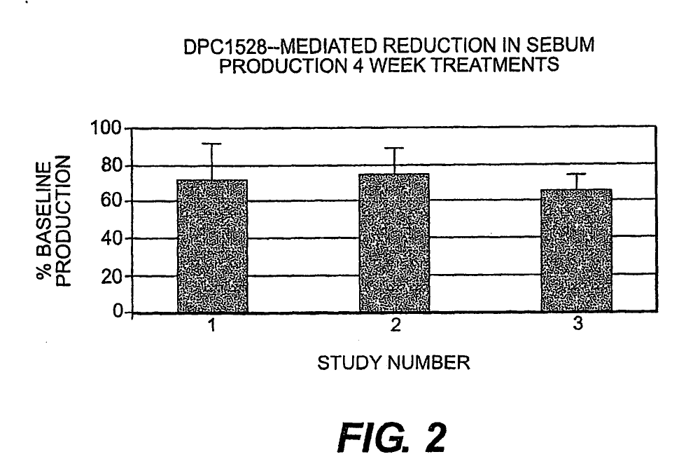

Three groups of 5 subjects each were used to evaluate the effect on sebum secretion rate of twice daily treatments with formulations containing 1 μM (Study 1 and 2) or 5 uM (Study 3) anti-HS5αR anti-sense oligonucleotide (DPC1528). Prior to treatment, each subject's rate of sebum secretion was monitored using a SebuMeter SM810 (Courage & Khazaka Electronics). For SebuMeter readings, the subjects were situated in an environmentally controlled room (temperature and humidity) for 30 minutes. Their foreheads were first swabbed to remove existing sebum, and the amount of new sebum secreted was measured. Subjects applied about 0.5 ml of a test formulation twice daily to their foreheads over a period of 4 weeks and their sebum secretion rates were then remeasured by SebuMeter. DPC1528 produced a 24-27% decrease in sebum production and 5 uM produced a 33% decrease (see FIG. 2).

### 8.6 Example 6

### Treatment of Subjects with an Anti-Sense Oligonucleotide to Assess Effects on Sebum Secretion

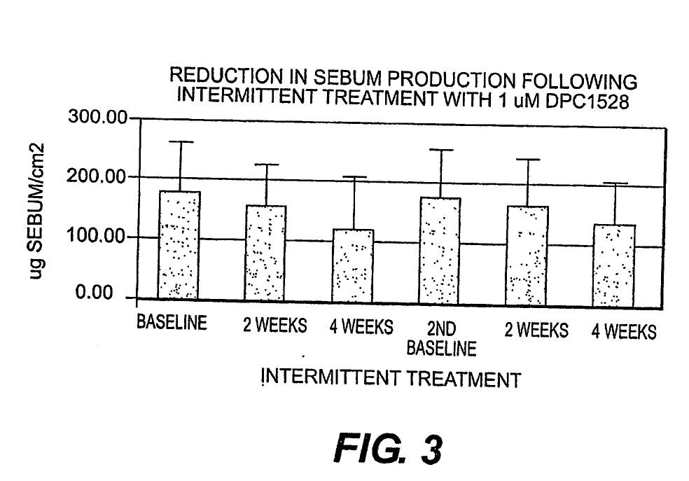

Five subjects were used to evaluate the effect of two rounds of twice daily treatments with 1 μM DPC1528. Prior to treatment, each subject's rate of sebum secretion was monitored using a SebuMeter SM810 (Courage & Khazaka Electronics). For SebuMeter reading the subjects were situated in an environmentally controlled room (temperature and humidity) for 30 minutes. Their foreheads were first swabbed to remove existing sebum, and the amount of new sebum secreted was then measured. Subjects applied about 0.5 ml of the DPC1528 solution twice daily to their foreheads over a period of 4 weeks, and then their sebum secretion rate was retaken by SebuMeter. 1 uM DPC1528 produced a 24% decrease in sebum secretion rate after the first 4 weeks. Sebum secretion returned to its pre-treatment level during a two-week period following cessation of treatment, and a second treatment period produced a 20% decrease in sebum secretion rate after 4 weeks of treatment (see FIG. 3).

### 8.7 Example 7

### Treatment of Subjects with an Anti-Sense Oligonucleotide to Assess Effects on Alopecia

Five subjects are used to evaluate the effect of two rounds of twice daily treatments with 1 μM DPC1676. Prior to treatment, each subject's degree of alopecia is measured by monitoring the number, length, and density of hair follicles by manual counting, or through counting in photomicrographs. Subjects apply about 0.5 ml of the DPC1676 solution twice daily to their scalp over a period of 4 weeks, and subsequently hair density is again monitored by the number, length, and density of hair follicles by manual counting, or through counting in photomicrographs, which demonstrates that there is an impact by the administration of DPC1676 on hair loss.

### 8.8 Example 8

### Assessment of Topical Delivery Vehicles

To evaluate the topical delivery vehicles of the invention, a study was performed using human cadaver skin explants to evaluate the vehicles' ability to enhance the penetration of oligonucleotides through the stratum corneum following topical applications. For this study, a phosphorothioate oligonucleotide comprised of fifteen nucleotides was chosen as the test compound. Following synthesis using standard hydrogen phosphonate chemistries, each inter-sugar linkage was reacted with elemental [³⁵S₈] which produced an oligonucleotide containing a radioactive sulfur molecule at each linkage. Using this oligonucleotide, the amount of oligonucleotide in the various layers of the skin can be quantified and its penetration into the various layers assessed. Whether or not the radiolabel is derived from metabolic breakdown and release of the radioactivity can also be determined. A stock solution of [³⁵S]-labeled oligonucleotide was prepared in Lipofectin™ (LTI, Gaithersburg, Md.) at a concentration of 7 μM. Lipofectin is comprised of a 1:1, (w/w) mixture of the cationic lipid N-[1-(2,3-dioleyoxyl)propyl]-N,N,N triethlyammonium chloride (DOTMA) and the neutral lipid dioleoyl phosphatidylethanolamine (DOPE). The positively charged and neutral lipids form liposomes that complex with nucleic acids and facilitate delivery of the nucleic acids into cells. In addition to the Lipofectin mixture, a similar concentration (7 uM) was prepared in 20% ethyl alcohol, 2% propylene glycol, 0.5% dimethyl isosorbide, 1% Laureth-4, 0.15% disodium EDTA, 0.03% pantethine in water to yield 100%.

Cryopreserved human scalp skin from a 62-year old Caucasian male was obtained from the Keystone Skin Bank organ procurement center. Serologies for this donor were negative. Human skin was clipped and shaved of hair, and the fat was removed from the dermal surface. Full-thickness disks were then made from the human skin with a number 11 cork boring tool (providing a 9-mm diameter, or a 63.6 mm², exposure area) and stored in ice cold Hank's balanced salt solution (HBSS). Skin samples were placed in a Bronaugh flow-through diffusion cell with the epidermal surface facing the upper chamber. The barrier function of each skin sample was determined by measuring the permeation of tritiated water over a 20 minute period. Each sample exhibited appropriate barrier function prior to application of test compound.

The experiment was conducted by application of a radiolabeled oligonucleotide in one of the above vehicles for 24 hours (N=6). After skin samples with sufficient barrier function were isolated, test compound (50 microliters of 7 μM oligonucleotide) was added to the epidermal surface of all skin samples. At the end of the incubation period, each skin surface was wiped with several dry cotton swabs and washed twice with detergent (1%). The skin was then removed from each Bronaugh cell and swabbed dry. The cotton swabs and wash solutions collected from the skin surface were dissolved in 1 N NaOH for at least 24 hours before radioassay. After washing, skin samples used in the penetration experiment were removed and separated into component layers. The stratum corneum of intact human skin was removed by tape-stripping and the epidermis and dermis were separated by heat treatment according to Bronaugh and Maibach (1991). The separated layers of human skin and the whole skin samples were digested in 1 N NaOH for at least 24 hours, then scintillation fluid was added to the samples, and they were placed in the dark at room temperature for at least 24 hours to allow chemoluminescence to decay. All of these fractions (remaining test article from cotton swabs, washes, skin layers, and receptor fluids collections) were evaluated by liquid scintillation spectrometry to determine the percent of radioactivity in each fraction and the amount of chemical absorbed across the skin surface (expressed as a percentage of the total recovered dose). The total absorbed value was calculated by adding the total radioactivity of the receptor fluid to the amount of chemical recovered from all of the skin layers. The percentage absorbed into the epidermis and dermis was calculated by dividing the total absorbed value by the total applied dose recovered from the system. Results showed that while oligonucleotide in Lipofectin did exhibit a limited ability to cross the stratum corneum, the dimethyl isosorbide vehicle resulted in enhanced penetration of oligonucleotide, as more than 13% of the applied oligonucleotide penetrated into the epidermis after 72 hours and more than 11% reached the dermis (see Table 5 below).

TABLE 5

| Formula: | dimethyl isosorbide vehicle | Lipofectin |
|---|---|---|
| Removed from surface: | 81.2 + 11.3 | 96.4 + 4.9 |
| Stratum corneum: | 5.2 + 1.4 | 1.1 + 0.5 |
| Epidermis: | 12.0 + 4.0 | 1.8 + 0.3 |
| Dermis: | 1.3 + 0.2 | 0.5 + 0.2 |
| Flow through: | 0.3 + 0.2 | 0.0 + 0.0 |

The numbers above represent the mean + SD (n = 6) as a % of the applied compound.

### 8.9 Example 9

### Effect of Dimethyl Isosorbide in Delivery Vehicles

Because a single application of oligonucleotide in the dimethyl isosorbide-containing vehicle exhibited an increased penetration into the epidermis, a study with repeat applications was conducted to further evaluate its use in topical delivery vehicles. To characterize the effect of multiple doses of a dimethyl isosorbide vehicle on skin penetration enhancement, a stock solution of [³⁵S]-labeled oligonucleotide was prepared in water at a concentration of 60 μM. Three hundred microliters of stock oligonucleotide was combined with 2.4 ml vehicle comprised of 20% ethyl alcohol, 2% propylene glycol, 0.5% dimethyl isosorbide, 1% Laureth-4, 0.15% disodium EDTA, 0.03% pantethine in water to yield a final concentration of 7 μM oligonucleotide. Fifty microliters of diluted solution to be used for application to the skin explants was shown to contain 83,751.64 dpm via liquid scintillation counting.

The experiment was conducted as three assay sets harvested at 24, 48, and 72 hours, respectively, each with N=6 disks for analysis. Skin from the same individual described above was characterized for barrier function and then used in the following experiment. After skin samples with sufficient barrier function were isolated, test compound (50 microliters of 7 μM oligonucleotide) was added to the epidermal surface of all skin samples. Receptor fluids (media flowing across the lower surface of the skin comprised of Hank's balanced salt solution containing 4% albumin) were collected at T=12 and 24 hours and Assay Set 1 was harvested at 24 hours for the respective analyses. The remaining two sets were washed with water to remove residual test composition, then the test composition was reapplied. Receptor fluids were collected from all remaining samples at T=36 and 48 hours and Assay Set 2 was harvested at 48 hours for the respective analyses. Assay Set 3 was washed with water to remove residue residual test composition, and the test composition was then reapplied. Receptor fluids were collected from all remaining samples at T=60 and 72 hours, and Assay Set 3 was harvested at 72 hours for the respective analyses.

At the end of the incubation period, each skin surface was wiped with several dry cotton swabs and washed twice with detergent (1%). The skin was then removed from each Bronaugh cell and swabbed dry. The cotton swabs and wash solutions collected from the skin surface were dissolved in 1 N NaOH for at least 24 hours before radioassay. After washing, skin samples used in the penetration experiment were removed and separated into component layers. The stratum corneum of intact human skin was removed by tape-stripping and the epidermis and dermis were separated by heat treatment according to Bronaugh Maibach (1991). The separated layers of human skin and the whole skin samples were digested in 1 N NaOH for at least 24 hours, then scintillation fluid was added to the samples, and they were placed in the dark at room temperature for at least 24 hours to allow chemoluminescence to decay. All of these fractions (remaining test article from cotton swabs, washes, skin layers, and receptor fluids collections) were evaluated by liquid scintillation spectrometry to determine the percent of radioactivity in each fraction and the amount of chemical absorbed across the skin surface (expressed as a percentage of the total recovered dose). The total absorbed value was calculated by adding the total radioactivity of the receptor fluid to the amount of chemical recovered from all of the skin layers. The percentage absorbed was calculated by dividing the total absorbed value by the total applied dose recovered from the system.

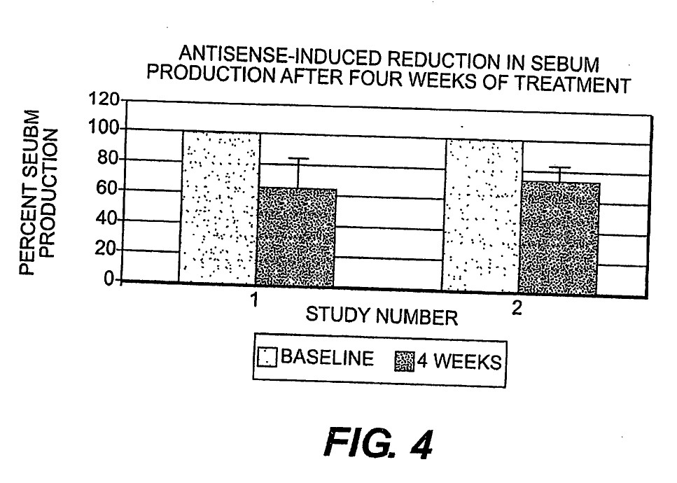

Results (FIG. 4) showed that with increasing time, there is an increase in the amount of oligonucleotide crossing the stratum corneum, saturating the epidermis, and that the compound is then able to penetrate the dermal layer at significant concentrations. Following application for 72 hours to human skin explants, the percentage of applied compound to reach the various layers in skin was 4.1% to the stratum corneum, 13.9% to the epidermis, and 11.6% to the dermis. The majority of compound (68.7%) was not absorbed into the explant and was recovered in washing steps prior to analysis of the various skin layers. Of the applied compound, only 1.7% penetrated through the skin explant and entered the support flow-through fluid.

Following treatment with the test compound, two skin plugs were separated into component parts and the dermal layers were analyzed for the characteristics of the radiolabel found in the dermal layer. Samples were digested with proteases and then the nucleic acids extracted with phenol and ethanol precipitated according to procedures known in the art. Nucleic acid samples were analyzed using denaturing polyacrylamide electrophoresis (20% polyacrylamide in 7 M urea) and autoradiographs were taken following four weeks exposure. Densitometric analysis of the autoradiography showed that the majority of the oligonucleotide compound extracted from the dermis was full-length when compared to the starting material and a ladder of compounds of similar lengths. The percent that was full-length was about 91% after 24 hours, about 84% at 48 hours and about 79% after 72 hours, as would be expected for a phosphorothioate-modified oligonucleotide in the presence of human nucleases. Thus, the penetration of radiolabel is reflective of the penetration of the oligonucleotide into the sub-stratum corneum layers.

Test flux rates were calculated for each collection interval and percent absorbed values were calculated for each 24 hour incubation period and used to calculate the concentration of compound deposited in the epidennis and dermis (see Table 6 below). The final flux data (ng/cm² hour for flux) were analyzed to determine “outliers,” replicates whose values were outside of 90% confidence limit (Schelfier, W C 1991) and these were eliminated. Descriptive statistic (mean and standard deviation) of all replicates not excluded as outliers were then calculated. Results showed that only a minor amount of the compound crossed into the flowthrough and that this deposition was time dependent in accordance with the increasing amounts in the dermis.

TABLE 6
Mean flux rates of [³⁵S] oligonucleotide delivered in vehible
after various times post application to intact human skin

| Time Period (hrs) | (ng/cm2/hr) |
|---|---|
| 0-12 | 0.032 +/− 0.123 |
| 12-24 | 0.160 +/− 0.325 |
| 24-36 | 0.460 +/− 0.569 |
| 36-48 | 0.784 +/− 0.883 |
| 48-60 | 0.932 +/− 0.782 |
| 60-72 | 1.263 +/− 1.231 |

From the above experiments, it is possible to determine that the penetration of oligonucleotides across the stratum corneum is not greatly enhanced by the use of the cationic liposomal formulation, Lipofectn. However, the combination of oligonucleotides with the vehicle containing dimethyl isosorbide, ethyl alcohol, glycerin, polyethylene glycol ester, propylene glycol, pantethine, zinc, and disodium EDTA produces a significant increase in penetration across the stratum corneum. Oligonucleotide applied using this vehicle, was able to produce a concentration of approximately 330 nM within the dermis. For nucleic acid-based compounds used in therapeutic applications, this would be a sufficient concentration to elicit an effect, as the IC₅₀ for most true anti-sense or ribozyme approaches is less that 30 nM.

Although the present invention has been described in detail with reference to examples above, it is understood that various modifications can be made without departing from the spirit of the invention. Accordingly, the invention is limited only by the following claims. All cited patents, patent applications, patent publications, and literature articles referred to in this application are expressly incorporated by reference in their entirety.

What is claimed:

1. A pharmaceutical composition comprising a therapeutically effective amount of at least one anti-sense oligonucleotide or a pharmaceutically acceptable salt, solvate, hydrate, clathrate, polymorph, or prodrug thereof, which substantially inhibits the expression of human steroid 5α-reductase type 1 in a patient, wherein the antisense oligonucleotide is complementary to and specifically hybridizes with at least a portion of a nucleotide sequence that encodes the protein designated as human steroid 5α-reductase type 1.

2. A pharmaceutical composition comprising a therapeutically effective amount of at least one anti-sense oligonucleotide or a pharmaceutically acceptable salt, solvate, hydrate, clathrate, polymorph, or prodrug thereof, which substantially inhibits the expression of human steroid 5α-reductase type 2 in a patient, wherein the antisense oligonucleotide is complementary to and specifically hybridizes with at least a portion of a nucleotide sequence that encodes the protein designated as human steroid 5α-reductase type 2.

3. The pharmaceutical composition of claim 1 or 2 further comprising a pharmaceutically acceptable carrier, vehicle, or excipient.

4. The pharmaceutical composition of claim 1 or 2, which is suitable for topical, intravenous, oral, or intranasal administration.

5. The pharmaceutical composition of claim 4, wherein the administration is topical.

6. The pharmaceutical composition of claim 1 or 2 further comprising a delivery formulation, which enhances the penetration of the anti-sense oligonucleotide across the stratum corneum of the skin.

7. The pharmaceutical composition of claim 6, wherein the delivery formulation comprises ethyl alcohol, propylene glycol, glycerin, dimethyl isosorbide, polyethylene glycol ester, EDTA, pantethine and a divalent cation.

8. The pharmaceutical composition of claim 7, wherein the delivery formulation comprises about 15 to about 40% ethyl alcohol; about 0.5 to about 5.0% propylene glycol; about 0.5 to about 5.0% glycerin; about 0.1 to about 2.0% dimethyl isosorbide; about 0.1 to about 2.0% polyethylene glycol ester; about 0.01 to about 0.5% disodium EDTA; about 0.01 to about 0.2% pantethine and about 0.01 to about 2% divalent cation.

9. The pharmaceutical composition of claim 5, wherein the composition is formulated for topical administration for a period of about one week.

10. The pharmaceutical composition of claim 9, wherein the composition is formulated for administration about once a day.

11. The pharmaceutical composition of claim 1, which is useful in treating or preventing skin inflammation, disorders related to excess sebum secretion, steatoma, cystic acne, excess keratin production, comedones, papule, milia, seborrheic dermatitis, dandruff, seborrheic eczema, infantile seborrheic eczema, seborrheic keratosis, rosacea, perioral dermatitis, sebaceous cysts, acne vulgaris, oily skin, seborrheic wart, senile wart, basil cell papilloma, hirsutism, dermatosis papulosa nigra.

12. The pharmaceutical composition of claim 2, which is useful in treating or preventing benign prostatic hyperplasia, prostate cancer, urinary incontinence, androgenic alopecia, and male pattern baldness.

13. The pharmaceutical composition of claim 1, wherein the antisense oligonucleotide specifically hybridizes to an mRNA transcript that encodes the protein designated as human steroid 5α-reductase type 1.

14. The pharmaceutical composition of claim 1, wherein the antisense oligonucleotide specifically hybridizes to a translation intitiation site, a 5′-untranslated sequences, 3′-untranslated sequences, any of the intron/exon junctions, or an intervening sequence of the mRNA transcript that is encoded by human steroid 5α-reductase type 1.

15. The pharmaceutical composition of claim 1, wherein the antisense oligonucleotide specifically hybridizes to a 5′ cap site or a region adjacent to a 5′ cap site of the human steroid 5α-reductase type 1 transcript.

16. The pharmaceutical composition of claim 1, wherein the antisense oligonucleotide specifically hybridizes to a portion of the coding sequence within the human steroid 5α-reductase type 1 mRNA transcript.

17. The pharmaceutical composition of claim 2, wherein the antisense oligonucleotide is complementary to and specifically hybridizes with at least a portion of a nucleotide sequence that encodes the protein designated as human steroid 5α-reductase type 2.

18. The pharmaceutical composition of claim 2, wherein the antisense oligonucleotide specifically hybridizes to an mRNA transcript that encodes the protein designated as human steroid 5α-reductase type 2.

19. The pharmaceutical composition of claim 2, wherein the antisense oligonucleotide specifically hybridizes to a translation intitiation site, a 5′-untranslated sequences, 3′-untranslated sequences, any of the intron/exon junctions, or an intervening sequence of the mRNA transcript that is encoded by human steroid 5α-reductase type 2.

20. The pharmaceutical composition of claim 2, wherein the antisense oligonucleotide specifically hybridizes to a 5′ cap site or a region adjacent to a 5′ cap site of the human steroid 5α-reductase type 2 transcript.

21. The pharmaceutical composition of claim 2, wherein the antisense oligonucleotide specifically hybridizes to a portion of the coding sequence within the human steroid 5α-reductase type 2 mRNA transcript.

22. The pharmaceutical composition of claim 1, wherein the anti-sense oligonucleotide comprises a sequence of at least 8 contiguous nucleotides selected from the group consisting of the complement of nucleotides 1-75 of SEQ ID NO: 1, the complement of nucleotides 620-682 of SEQ ID NO: 1 and the complement of nucleotides 1175-1250 of SEQ ID NO: 1.

23. The pharmaceutical composition of claim 1, wherein the anti-sense oligonucleotide comprises a sequence of at least 8 contiguous nucleotides selected from the group consisting of SEQ ID NOS: 2, 3, 4, 5, 6, 7, 8, 9, 10 and 11.

24. The pharmaceutical composition of claim 2, wherein the anti-sense oligonucleotide comprises a sequence of at least 8 contiguous nucleotides selected from the group consisting of SEQ ID NOS: 37, 38, 39, 40, 41, 42, 43, 44, 45, and 46.

25. The pharmaceutical composition of claim 1 or 2 further comprising a second active agent, wherein the second active agent is an antifungal agent, an H¹ receptor antagonist, a retinoid, an anti-obesity drug, a hormone, a phosphodiesterase-5 inhibitor, an antibiotic, an anti-cancer agent, a topical steroid, or an astringent.

26. A method of treating or preventing a disorder that can be treated or prevented by inhibiting the conversion of testosterone to dihydrotestosterone, which comprises administering to a patient in need thereof a therapeutically effective amount of at least one anti-sense oligonucleotide or a pharmaceutically acceptable salt, solvate, hydrate, clathrate, polymorph, or prodrug thereof, which substantially inhibits the expression of human steroid 5α-reductase type 1 in a patient.

27. A method of treating or preventing skin inflammation, disorders related to excess sebum secretion, steatoma, cystic acne, excess keratin production, comedones, papule, milia, seborrheic dermatitis, seborrheic eczema, infantile seborrheic eczema, seborrheic keratosis, rosacea, perioral dermatitis, sebaceous cysts, acne vulgaris, oily skin, seborrheic wart, senile wart, basil cell papilloma, hirsutism, dermatosis paulosa nigra, benign prostatic hyperplasia, prostate cancer, urinary incontinence, androgenic alopecia, and male pattern baldness in a patient in need thereof, which comprises administering to said patient a therapeutically effective amount of at least one anti-sense oligonucleotide or a pharmaceutically acceptable salt, solvate, hydrate, clathrate, polymorph, or prodrug thereof, which substantially inhibits the expression of human steroid 5α-reductase type 1 in a patient.

28. A method of treating or preventing a disorder that can be treated or prevented by inhibiting the conversion of testosterone to dihydrotestosterone, which comprises administering to a patient in need thereof a therapeutically effective amount of at least one anti-sense oligonucleotide or a pharmaceutically acceptable salt, solvate, hydrate, clathrate, polymorph, or prodrug thereof, which substantially inhibits the expression of human steroid 5α-reductase type 2 in a patient.

29. A method of treating or preventing skin inflammation, disorders related to excess sebum secretion, steatoma, cystic acne, excess keratin production, comedones, papule, milia, seborrheic dermatitis, seborrheic eczema, infantile seborrheic eczema, seborrheic keratosis, rosacea, perioral dermatitis, sebaceous cysts, acne vulgaris, oily skin, seborrheic wart, senile wart, basil cell papilloma, hirsutism, dermatosis paulosa nigra, benign prostatic hyperplasia, prostate cancer, urinary incontinence, androgenic alopecia, and male pattern baldness in a patient in need thereof, which comprises administering to said patient a therapeutically effective amount of at least one anti-sense oligonucleotide or a pharmaceutically acceptable salt, solvate, hydrate, clathrate, polymorph, or prodrug thereof, which substantially inhibits the expression of human steroid 5α-reductase type 2 in a patient.

30. The method of claim 26, 27, 28, or 29, wherein the anti-sense oligonucleotide binds to a region of the human steroid 5α-reductase type 1 transcript selected from the group consisting of: the translation initiation site, a region 5′ to the translation initiation site, a region 3′ to the translation initiation site, the 5′ cap region of the mRNA, a region 5′ to the cap region of the mRNA, a region 3′ to the cap region of the mRNA and the 3′ untranslated region.

31. The method of claim 26, 27, 28, or 29, wherein the anti-sense oligonucleotide comprises a sequence of at least about 8 contiguous nucleotides selected from the group consisting of the complement of nucleotides 1-75 of SEQ ID NO: 1, the complement of nucleotides 620-682 of SEQ ID NO: 1 and the complement of nucleotides 1175-1250 of SEQ ID NO: 1.

32. The method of claim 26, 27, 28, or 29, wherein the anti-sense oligonucleotide is about 8 to about 50 nucleotides in length.

33. The method of claim 26, 27, 28, or 29, wherein the anti-sense oligonucleotide comprises a sequence of at least about 8 contiguous nucleotides selected from the group consisting of SEQ ID NOS: 2, 3, 4, 5, 6, 7, 8, 9, 10 and 11.

34. The method of claim 26, 27, 28, or 29, wherein the composition further comprises a second molecule which substantially inhibits the activity of human steroid 5α-reductase type 1.

35. The method of claim 26, 27, 28, or 29, wherein the composition is applied to the skin over a period of about one week.

36. The method of claim 15, wherein the composition is applied to the skin about once per day.

37. A dermatological composition for treating or preventing a disorder of the skin comprising at least one anti-sense oligonucleotide which substantially inhibits the expression of human steroid 5α-reductase type 1 and further comprising an agent to enhance the penetration of the anti-sense oligonucleotide across the stratum corneum

38. The dermatological composition of claim 37, wherein the agent to enhance the penetration is ethyl alcohol, propylene glycol, glycerin, dimethyl isosorbide, polyethylene glycol ester, EDTA, pantethine and a divalent cation.

39. The dermatological composition of claim 38, wherein the anti-sense oligonucleotide comprises a sequence of at least 8 contiguous nucleotides selected from the group consisting of the complement of nucleotides 1-75 of SEQ ID NO: 1, the complement of nucleotides 620-682 of SEQ ID NO: 1 and the complement of nucleotides 1175-1250 of SEQ ID NO: 1.

40. The dermatological composition of claim 38, wherein the anti-sense oligonucleotide comprises a sequence of at least 8 contiguous nucleotides selected from the group consisting of SEQ ID NOS: 2, 3, 4, 5, 6, 7, 8, 9, 10 and 11.

41. The dermatological composition of claim 38, wherein the composition further comprises a second agent, which inhibits the blockage of skin pores by sebaceous material.

42. The method of claim 41, wherein the second molecule is retinoic acid, tretinoin, or retin-A.

43. A pharmaceutical composition comprising a therapeutically effective amount of at least one ribozyme or siRNA or a pharmaceutically acceptable salt, solvate, hydrate, clathrate, polymorph, or prodrug thereof, which substantially inhibits the expression of human steroid 5α-reductase type 1 in a patient.

44. A pharmaceutical composition comprising a therapeutically effective amount of at least one ribozyme or siRNA or a pharmaceutically acceptable salt, solvate, hydrate, clathrate, polymorph, or prodrug thereof, which substantially inhibits the expression of human steroid 5α-reductase type 2 in a patient.

45. The pharmaceutical composition of claim 43 or 44 further comprising a pharmaceutically acceptable carrier, vehicle, or excipient.

46. The pharmaceutical composition of claim 43 or 44, which is suitable for topical, intravenous, oral, or intranasal administration.

47. The pharmaceutical composition of claim 4, wherein the administration is topical.

48. The pharmaceutical composition of claim 43 or 44 further comprising a delivery formulation, which enhances the penetration of the anti-sense oligonucleotide across the stratum corneum of the skin.

49. The pharmaceutical composition of claim 48, wherein the delivery formulation comprises ethyl alcohol, propylene glycol, glycerin, dimethyl isosorbide, polyethylene glycol ester, EDTA, pantethine and a divalent cation.

50. The pharmaceutical composition of claim 49, wherein the delivery formulation comprises about 15 to about 40% ethyl alcohol; about 0.5 to about 5.0% propylene glycol; about 0.5 to about 5.0% glycerin; about 0.1 to about 2.0% dimethyl isosorbide; about 0.1 to about 2.0% polyethylene glycol ester; about 0.01 to about 0.5% disodium EDTA; about 0.01 to about 0.2% pantethine and about 0.01 to about 2% divalent cation.

51. The pharmaceutical composition of claim 48, wherein the composition is formulated for topical administration for a period of at least four weeks.

52. The pharmaceutical composition of claim 51, wherein the composition is formulated for administration twice a day.

53. The pharmaceutical composition of claim 43, which is useful in treating or preventing skin inflammation, disorders related to excess sebum secretion, steatoma, cystic acne, excess keratin production, comedones, papule, milia, seborrheic dermatitis, dandruff, seborrheic eczema, infantile seborrheic eczema, seborrheic keratosis, rosacea, perioral dermatitis, sebaceous cysts, acne vulgaris, oily skin, seborrheic wart, senile wart, basil cell papilloma, hirsutism, dermatosis papulosa nigra.

54. The pharmaceutical composition of claim 44, which is useful in treating or preventing benign prostatic hyperplasia, prostate cancer, urinary incontinence, androgenic alopecia, and male pattern baldness.

55. The pharmaceutical composition of claim 43, wherein the ribozyme specifically hybridizes to an mRNA transcript that encodes the protein designated as human steroid 5α-reductase type 1.

56. The pharmaceutical composition of claim 43, wherein the ribozyme specifically hybridizes to a translation intitiation site, a 5′-untranslated sequences, 3′-untranslated sequences, any of the intron/exon junctions, or an intervening sequence of the mRNA transcript that is encoded by human steroid 5α-reductase type 1.

57. The pharmaceutical composition of claim 43, wherein the ribozyme specifically hybridizes to a 5′ cap site or a region adjacent to a 5′ cap site of the human steroid 5α-reductase type 1 transcript.

58. The pharmaceutical composition of claim 44, wherein the ribozyme specifically hybridizes to a portion of the coding sequence within the human steroid 5α-reductase type 1 mRNA transcript.

59. The pharmaceutical composition of claim 44, wherein the ribozyme is complementary to and specifically hybridizes with at least a portion of a nucleotide sequence that encodes the protein designated as human steroid 5α-reductase type 2.

60. The pharmaceutical composition of claim 44, wherein the ribozyme specifically hybridizes to an mRNA transcript that encodes the protein designated as human steroid 5α-reductase type 2.

61. The pharmaceutical composition of claim 44, wherein the ribozyme specifically hybridizes to a translation intitiation site, a 5′-untranslated sequences, 3′-untranslated sequences, any of the intron/exon junctions, or an intervening sequence of the mRNA transcript that is encoded by human steroid 5α-reductase type 2.

62. The pharmaceutical composition of claim 44, wherein the ribozyme specifically hybridizes to a 5′ cap site or a region adjacent to a 5′ cap site of the human steroid 5α-reductase type 2 transcript.

63. The pharmaceutical composition of claim 44, wherein the ribozyme specifically hybridizes to a portion of the coding sequence within the human steroid 5α-reductase type 2 mRNA transcript.

64. The pharmaceutical composition of claim 43 or 44 further comprising a second active agent.

65. The pharmaceutical composition of claim 43 or 44 wherein the second active agent is an antifungal agent, an H¹ receptor antagonist, a retinoid, an anti-obesity drug, a hormone, a phosphodiesterase-5 inhibitor, an antibiotic, an anti-cancer agent, a topical steroid, or an astringent.


SEQUENCE LISTING (36 sequences)

SEQ ID NO: 1 - DNA, 2102 bp, Homo sapiens
  misc_feature: Human steroid 5alpha-reductase type 1
gggcatggagcacgctgcccagccctggcgatggcaacggcgacgggggtggcggaggagcgcctgctggccgcgctcgcctacctgcagtgcgccgtgggctgcgcggtcttcgcgcggaatcgtcagacgaactcagtgtacggccgccacgcgctgcccagccacaggctccgagtgccggcgcgggccgcctgggtggtgcaggagctgccctcgctggccctgccgctctaccagtacgccagcgagtccgccccgcgtctccgcagcgcgcccaactgcatcctcctggccatgttcctcgtccactacgggcatcggtgcttaatttacccgtttctgatgcgaggaggaaagcctatgccactgttggcatgtacaatggcgattatgttctgtacctgtaacggctatttgcaaagcagatacttgagccattgtgcagtgtatgctgatgactgggtaacagatccccgttttctaataggttttggcttgtggttaacaggcatgttgataaacatccattcagatcatatcctaaggaatctcagaaaaccaggagatactggatacaaaataccaaggggaggcttatttgaatacgtaactgcagccaactattttggagaaatcatggagtggtgtggctatgccctggccagctggtctgtccaaggcgcggcttttgctttcttcacgttttgttttttatctggtagagcaaaagagcatcatgagtggtacctccggaaatttgaagagtatccaaagttcagaaaaattataattccatttttgttttaagtgcgtttttcatgaaattatcttcaacttgaagctttccaatggcgcttctctatggactttgtaaataagttatatctttgtaattttcctgctactttatcattttcaagatgtcctctaggaattttttttctagtaattttgcaatctacctaataagtacctaaatacgctgaaatggaggttgaatatcctactgtgtaacaggtcagaatttcaagctctgggtaataactgctgatattttttctaatttcaaatttacctcttttggctatgtcttgccaagtgtgtatgagactagactttacaactgtctttgatggcattttcagaacaataaatgtcacaatcccttctatagccccctacagtgatctcttcaaggtcaactgcagtgttgcttccctccccctatagggctggaatctgtctaggagccctctctcggaggccacagaggctgggggtagccattgtgcagtcatggcccgggggaaacttgccaaccttcgtgtcaggtgctgtgtgtaagtggagaacttggggatagaggaggaagctcctcgtggcccttccaaggtgaggcaaaggcatctggacttgttccagcccagcccaccgggtgacatcaccgggcagggaggggtgctggtggtggttcatacggagtaagctgctctgcctgtgtgagtggctcctgggccctaaacaggcacctttaggccatgggtcactcaccgtgagccatcaatgtgctctggtctgacatggtttctctctgtcttctagtctagacctagtttttttgttctgttccccacgtatggatatagtagagattgttgtctgtgaaatttctcttttgtagattttgagttttcccttgtagtgtaaagaatgatcactttctgtaacaataacaagaccactttttaagatttatcctgtttgttctttgttgattgaaacataataattgttaaaattctctacagccttctttttcttccatagctaatcttccttctaatagtttttgctttctgttttgctgttgttgctttgcaaagctttcccctcatagcctgtacctgttatcaatataaaataatcttcctgttgaatgcttcatgacttgaattctactttgataaaaacattgccatactgctttttatcttgatgaattcatctggcattgctttgccttatcatctcatctggagtttttaaatgccatttgtttcagttgtctttaacaacataataaatagactttgccattt

SEQ ID NO: 2 - DNA, 21 bp, artificial
  Human steroid 5alpha-reductase type 1 anti-sense oligonucleotide
tgggcagcgtgctccatgccc

SEQ ID NO: 3 - DNA, 21 bp, artificial
  Human steroid 5alpha-reductase type 1 anti-sense oligonucleotide
cagggctgggcagcgtgctcc

SEQ ID NO: 4 - DNA, 21 bp, artificial
  Human steroid 5alpha-reductase type 1 anti-sense oligonucleotide
tgccatcgccagggctgggca

SEQ ID NO: 5 - DNA, 21 bp, artificial
  Human steroid 5alpha-reductase type 1 anti-sense oligonucleotide
ccccgtcgccgttgccatcgc

SEQ ID NO: 6 - DNA, 21 bp, artificial
  Human steroid 5alpha-reductase type 1 anti-sense oligonucleotide
tcgccgttgccatcgccaggg

SEQ ID NO: 7 - DNA, 21 bp, artificial
  Human steroid 5alpha-reductase type 1 anti-sense oligonucleotide
ggcgctcctccgccacccccg

SEQ ID NO: 8 - DNA, 21 bp, artificial
  Human steroid 5alpha-reductase type 1 anti-sense oligonucleotide
gacagaccagctggccagggc

SEQ ID NO: 9 - DNA, 21 bp, artificial
  Human steroid 5alpha-reductase type 1 anti-sense oligonucleotide
gcgccattggaaagcttcaag

SEQ ID NO: 10 - DNA, 21 bp, artificial
  Human steroid 5alpha-reductase type 1 anti-sense oligonucleotide
gcgtatttaggtacttattag

SEQ ID NO: 11 - DNA, 21 bp, artificial
  Human steroid 5alpha-reductase type 1 anti-sense oligonucleotide
tccagccctatagggggaggg

SEQ ID NO: 12 - RNA, 21 nt, artificial
  Human steroid 5alpha-reductase type 1 siRNA
cacugaguucgucugacgauu

SEQ ID NO: 13 - RNA, 22 nt, artificial
  Human steroid 5alpha-reductase type 1 siRNA
uacagaacaucaauucgccauu

SEQ ID NO: 14 - RNA, 21 nt, artificial
  Human steroid 5alpha-reductase type 1 siRNA
uacagaacaucaaucgccauu

SEQ ID NO: 15 - RNA, 21 nt, artificial
  Human steroid 5alpha-reductase type 1 siRNA
aguuggcugcaguuacguauu

SEQ ID NO: 16 - RNA, 21 nt, artificial
  Human steroid 5alpha-reductase type II siRNA
uguggaauccuguagcugauu

SEQ ID NO: 17 - RNA, 21 nt, artificial
  Human steroid 5alpha-reductase type II siRNA
cuucaggcucuccgugugcuu

SEQ ID NO: 18 - RNA, 21 nt, artificial
  Human steroid 5alpha-reductase type II siRNA
guagccuugaaggacuccauu

SEQ ID NO: 19 - RNA, 21 nt, artificial
  Human steroid 5alpha-reductase type II siRNA
guagcugauuucuccaggcuu

SEQ ID NO: 20 - RNA, 33 nt, artificial
  Human steroid 5alpha-reductase type I ribozyme
uccgcccugaugaguccgugaggacgaaacccc

SEQ ID NO: 21 - RNA, 34 nt, artificial
  Human steroid 5alpha-reductase type I ribozyme
cagccccugaugaguccgugaggacgaacacggc

SEQ ID NO: 22 - RNA, 33 nt, artificial
  Human steroid 5alpha-reductase type I ribozyme
gccguucugaugaguccgugaggacgaaacagg

SEQ ID NO: 23 - RNA, 33 nt, artificial; identical to SEQ ID NO: 22
  Human steroid 5alpha-reductase type I ribozyme

SEQ ID NO: 24 - RNA, 33 nt, artificial
  Human steroid 5alpha-reductase type II ribozyme
cacugacugaugaguccgugaggacgaaaccug

SEQ ID NO: 25 - RNA, 33 nt, artificial
  Human steroid 5alpha-reductase type II ribozyme
cgacgucugaugaguccgugaggacgaaacaag

SEQ ID NO: 26 - RNA, 33 nt, artificial
  Human steroid 5alpha-reductase type II ribozyme
gcaccacugaugaguccgugaggacgaaaccag

SEQ ID NO: 27 - RNA, 33 nt, artificial
  Human steroid 5alpha-reductase type II ribozyme
uguacccugaugaguccgugaggacgaaaccca

SEQ ID NO: 28 - DNA, 2437 bp, Homo sapiens
  Human steroid 5alpha-reductase type II
gcggccaccggcgaggaacacggcgcgatgcaggttcagtgccagcagagcccagtgctggcaggcagcgccactttggtcgcccttggggcactggccttgtacgtcgcgaagccctccggctacgggaagcacacggagagcctgaagccggcggctacccgcctgccagcccgcgccgcctggttcctgcaggagctgccttccttcgcggtgcccgcggggatcctcgcccggcagcccctctccctcttcgggccacctgggacggtacttctgggcctcttctgcgtacattacttccacaggacatttgtgtactcactgctcaatcgagggaggccttatccagctatactcattctcagaggcactgccttctgcactggaaatggagtccttcaaggctactatctgatttactgtgctgaataccctgatgggtggtacacagacatacggtttagcttgggtgtcttcttatttattttgggaatgggaataaacattcatagtgactatatattgcgccagctcaggaagcctggagaaatcagctacaggattccacaaggtggcttgtttacgtatgtttctggagccaatttcctcggtgagatcattgaatggatcggctatgccctggccacttggtccctcccagcacttgcatttgcatttttctcactttgtttccttgggctgcgagcttttcaccaccataggttctacctcaagatgtttgaggactaccccaaatctcggaaagcccttattccattcatcttttaaaggaaccaaattaaaaaggagcagagctcccacaatgctgatgaaaactgtcaagctgctgaaactgtaattttcatgatataatagtcatatatatatatatatatatatatatatatatatatatgtatatatgtaatagtaggtctcctggcgttctgccagctggcctggggattctgagtggtgtctgcttagagtttactcctacccttccagggacccctatcctgatccccaactgaagcttcaaaaagccacttttccaaatggcgacagttgcttcttagctattgctctgagaaagtacaaacttctcctatgtctttcaccgggcaatccaagtacatgtggcttcatacccactccctgtcaatgcaggacaactctgtaatcaagaattttttgacttgaaggcagtacttatagaccttattaaaggtatgcattttatacatgtaacagagtagcagaaatttaaactctgaagccacaaagacccagagcaaacccactcccaaatgaaaaccccagtcatggcttcctttttcttggttaattaggaaagatgagaaattattaggtagaccttgaatacaggagccctctcctcatagtgctgaaaagatactgatgcattgacctcatttcaaatttgtgcagtgtcttagttgatgagtgcctctgttttccagaagatttcacaatccccggaaaactggtatggctattcttgaaggccaggttttaataaccacaaacaaaaaggcatgaacctgggtggcttatgagagagtagagaacaacatgaccctggatggctactaagaggatagagaacagttttacaatagacattgcaaactctcatgtttttggaaactggtggcaatatccaaataatgagtagtgtaaaacaaagagaattaatgatgaggttacatgctgcttgcctccaccagatgtccacaacaatatgaagtacagcagaagccccaagcaactttcctttcctggagcttcttccttgtagttctcaggacctgttcaagaaggtgtctcctaggggcagcctgaatgcctccctcaaaggacctgcaggcagagactgaaaattgcagacagaggggcacgtctgggcagaaaacctgttttgtttggctcagacatatagtttttttttttttacaaagtttcaaaaacttaaaaatcaggagattccttcataaaactctagcattctagtttcatttaaaaagttggaggatctgaacatacagagcccacatttccacaccagaactggaactacgtagctagtaagcatttgagtttgcaaactcttgtgaaggggtcaccccagcatgagtgctgagatatggactctctaaggaaggggccgaacgcttgtaattggaatacatggaaatatttgtcttctcaggcctatgtttgcggaatgcattgtcaatatttagcaaactgttttgacaaatgagcaccagtggtactaagcacagaaactcactatataagtcacataggaaacttgaaaggtctgaggatgatgtagattactgaaaaatacaaattgcaatcatataaataagtgtttttgttgttcattaaatacctttaaatcatg

SEQ ID NO: 29 - RNA, 21 nt, artificial
  complement of siRNA inhibitor SEQ ID NO 12
ucgucagacgaacucaguguu

SEQ ID NO: 30 - RNA, 22 nt, artificial
  complement of siRNA inhibitor SEQ ID NO 13
uggcgaauugauguucuguauu

SEQ ID NO: 31 - RNA, 21 nt, artificial
  complement of siRNA inhibitor SEQ ID NO 14
uggcgauugauguucuguauu

SEQ ID NO: 32 - RNA, 21 nt, artificial
  complement of siRNA inhibitor SEQ ID NO 15
uacguaacugcagccaacuuu

SEQ ID NO: 33 - RNA, 21 nt, artificial
  complement of siRNA inhibitor SEQ ID NO 16
ucagcuacaggauuccacauu

SEQ ID NO: 34 - RNA, 21 nt, artificial
  complement of siRNA inhibitor SEQ ID NO 17
gcacacggagagccugaaguu

SEQ ID NO: 35 - RNA, 21 nt, artificial
  complement of siRNA inhibitor SEQ ID NO 18
uggaguccuucaaggcuacuu

SEQ ID NO: 36 - RNA, 21 nt, artificial
  complement of siRNA inhibitor SEQ ID NO 19
gccuggagaaaucagcuacuu